(19) United States
(12) Patent Application Publication
(10) Pub. No.: US 2019/0008574 A1
(43) Pub. Date: Jan. 10, 2019

(54) TEMPERATURE MEASUREMENT SYSTEMS, METHOD AND DEVICES

(71) Applicant: Securus Medical Group, Inc., Cleveland, OH (US)

(72) Inventors: Vincent Cangialosi, South Hamilton, MA (US); John T. Garibotto, Marblehead, MA (US); Jesse Jones, Woburn, MA (US); Elizabeth Begin, Billerica, MA (US)

(21) Appl. No.: 15/748,836

(22) PCT Filed: Aug. 11, 2016
(86) PCT No.: PCT/US2016/046526

§ 371 Date: Jan. 30, 2018

Related U.S. Application Data
(60) Provisional application No. 62/204,186, filed on Aug. 12, 2015.

(51) Int. Cl. A61B 18/12; A61B 5/00; A61B 5/01

(52) U.S. Cl. CPC A61B 18/12; A61B 5/0086; A61B 2018/00714; A61B 5/6847; A61B 5/01

(57) ABSTRACT

A system that produces temperature estimations of a tissue surface comprises a base including a motion unit. A fiber assembly includes at least one fiber constructed and arranged to receive infrared energy from the tissue surface, the fiber assembly transmissive of infrared energy; the fiber assembly including a proximal end, a distal end and a body. An optical element redirects received infrared energy to the distal end of the fiber optic. A linkage is coupled between the base and the optical element, the fiber extending through the linkage, the linkage coupled to the motion unit at a proximal end and the optical element at a distal end, the motion unit constructed and arranged to rotate the linkage about the fiber assembly to thereby rotate the optical element at the distal end.

### RELATED APPLICATIONS

This application claims priority to U.S. Provisional Application Ser. No. 62/204,186 filed Aug. 12, 2015 entitled “Temperature Measurement Systems, Method and Devices,” the content of which is incorporated by reference in its entirety.

This patent application is related to PCT/US15/33680 filed Jun. 2, 2015, which claims the benefit of U.S. Provisional Application Ser. No. 62/007,677 filed Jun. 4, 2014, and is a continuation-in-part (CIP) of International Patent Application Serial Number PCT/US2013/076961, entitled “Temperature Measurement Systems, Method and Devices,” filed Dec. 20, 2013, which in turn claims the benefit of U.S. Provisional Application Ser. No. 61/749,617 filed Jan. 7, 2013, the content of each of which is incorporated by reference in its entirety.

This patent application is related to International Patent Application Serial Number PCT/US2011/061802, entitled “Ablation and Temperature Measurement Devices”, filed Nov. 22, 2011 and U.S. Provisional Application Ser. No. 61/417,416, filed Nov. 27, 2010, and U.S. patent application Ser. No. 12/934,008 filed Sep. 22, 2010, the content of each of which is incorporated by reference in its entirety.

### FIELD

Embodiments relate generally to the field of tissue temperature monitoring, and more particularly, to ablation and temperature measurement devices and systems that monitor tissue temperature during energy delivery.

### BACKGROUND

Numerous medical procedures include the delivery of energy to change the temperature of target tissue, such as to ablate or otherwise treat the tissue. With today's energy delivery systems, it is difficult for an operator of the system, such as a clinician, to treat all of the target tissue while avoiding adversely affecting non-target tissue. In treatment of a cardiac arrhythmia, ablation of heart tissue can often ablate target tissue such as heart wall tissue, while inadvertently causing thermal damage to esophageal and other surrounding, non-target tissue. Similarly, in airway ablation for the treatment of COPD, asthma, tumors and other airway disorders the esophageal tissue may be inadvertently thermally damaged. In tumor ablation procedures, cancerous tissue ablation may also be incomplete or healthy tissue may be damaged.

There is a need for energy delivery and energy monitoring systems which allow a clinician to properly deliver energy to target tissue, while avoiding any destructive energy delivery to non-target tissue.

### SUMMARY

In an aspect, a system that produces temperature estimations of a tissue surface, comprises: a base including a motion unit; a fiber assembly including at least one fiber constructed and arranged to receive infrared energy from the tissue surface, the fiber assembly transmissive of infrared energy; the fiber assembly including a proximal end, a distal end and a body; an optical element that redirects received infrared energy to the distal end of the fiber optic; and a linkage coupled between the base and the optical element, the fiber extending through the linkage, the linkage coupled to the motion unit at a proximal end and the optical element at a distal end, the motion unit constructed and arranged to rotate the linkage about the fiber assembly to thereby rotate the optical element at the distal end.

In some embodiments, the linkage comprises a torque coil.

In some embodiments, the linkage comprises a longitudinal channel through which the fiber is positioned.

In some embodiments, the linkage comprises a woven fabric of material. In some embodiments, the material comprises at least one of wire, titanium wire, stainless steel wire, steel, alloy, graphite, composite, plastic, or a woven fabric of material.

In some embodiments, the linkage comprises an elongated tubular material that is torsionally rigid and longitudinally flexible.

In some embodiments, the linkage comprises laser-cut tubing.

In some embodiments, the optical element comprises a reflective surface.

In some embodiments, the reflective surface redirects infrared energy incident thereon toward the distal end of the fiber assembly.

In some embodiments, the reflective surface redirects infrared energy incident thereon in a direction transverse a longitudinal direction of the fiber assembly to the distal end of the fiber assembly in the longitudinal direction of the fiber assembly.

In some embodiments, the reflective surface is planar.

In some embodiments, the reflective surface is non-planar.

In some embodiments, the reflective surface comprises a convex profile.

In some embodiments, the reflective surface comprises a concave profile.

In some embodiments, the reflective surface comprises a profile defined by a relationship having an order greater than first order.

In some embodiments, the optical element further comprises a lens positioned between the reflective surface and the distal end of the fiber assembly.

In some embodiments, the reflective surface redirects infrared energy incident thereon toward the lens and wherein the lens focuses the redirected infrared energy toward the distal end of the fiber assembly.

In some embodiments, the system further comprises a holder at which the optical element including the reflective surface is positioned.

In some embodiments, the holder is coupled to the linkage at a proximal end and includes a longitudinal opening within which the reflective surface is positioned.

In some embodiments, the system further comprises a lens positioned in the longitudinal opening.

In some embodiments, the optical element comprises a reflective body and wherein infrared energy incident thereon reflects at the reflective surface substantially external to the reflective body.

In some embodiments, the optical element comprises a refractive body and wherein infrared energy incident thereon propagates through the refractive body.

In some embodiments, the reflective surface is positioned on an external surface of the refractive body and wherein the incident energy reflects internally relative to the reflective surface.

In some embodiments, a dual-holder includes an inner holder attached to a lens, and in a stationary position relative to the fiber assembly, the lens in a stationary position relative to a mirror of the optical element, the dual-holder further including an outer holder connected to the linkage.

In some embodiments, the system further comprises a lens positioned between the reflective surface of the optical element and the distal end of the fiber assembly.

In some embodiments, the lens is rotationally fixed wherein the optical element rotates relative to the lens.

In some embodiments, the system further comprises a first holder fixedly coupled to the distal end of the fiber assembly, wherein the lens is coupled to the holder.

In some embodiments, a distance between the distal end of the fiber assembly and the lens is fixed by the first holder.

In some embodiments, the system further comprises a second holder fixedly coupled to the linkage and at which the optical element including the reflective surface is positioned wherein the holder is coupled to the linkage at a proximal end and includes a longitudinal opening within which the reflective surface is positioned.

In some embodiments, the second holder rotates about the first holder.

In some embodiments, the system further comprises a bearing positioned between the distal end of the fiber assembly and the second holder.

In some embodiments, the system further comprises a bearing positioned between the first holder and the second holder.

In some embodiments, the system further comprises a holder fixedly coupled to the linkage and at which the optical element including the reflective surface is positioned, wherein the holder is coupled to the linkage at a proximal end and includes a longitudinal opening within which the reflective surface is positioned.

In some embodiments, the holder rotates about the distal end of the fiber assembly.

In some embodiments, the holder is coupled to the linkage so that the distal end of the fiber assembly is positioned at a first position of the holder and the optical element is positioned at a second position of the holder, the second position being spaced apart from the first position.

In some embodiments, the holder further comprises an end cap at a distal end of the longitudinal opening, opposite the first position.

In some embodiments, a first portion of the end cap is positioned within the longitudinal opening and a second portion of the end cap extends beyond a distal end of the longitudinal opening.

In some embodiments, the second portion of the end cap has an end surface that lies at an acute angle relative to a longitudinal axis of the longitudinal opening of the holder.

In some embodiments, the reflective surface of the optical element lies at an acute angle relative to a longitudinal axis of the longitudinal opening of the holder and wherein the reflective surface abuts the end surface of the second portion of the end cap.

In some embodiments, the end cap has a rounded outer profile.

In some embodiments, the holder further comprises a lateral opening extending from the longitudinal opening through a sidewall of the holder.

In some embodiments, the system further comprises a lens positioned in the lateral opening.

In some embodiments, the system further comprises a protective sleeve positioned about the sidewall of the holder and covering the lateral opening.

In some embodiments, the reflective surface of the optical element lies at an acute angle relative to a longitudinal axis of the longitudinal opening of the holder.

In some embodiments, the system further comprises a bearing positioned between the body of the fiber assembly and the linkage.

In some embodiments, the bearing comprises an elongated lubricious sleeve.

In some embodiments, the bearing comprises a slip ring.

In some embodiments, the fiber assembly is rotationally fixed relative to the linkage and the motion unit.

In some embodiments, the motion unit is constructed and arranged to translate the fiber assembly along a translational axis relative to the base.

In some embodiments, the motion unit is constructed and arranged to translate the linkage and optical element along a translational axis relative to the base.

In some embodiments, the motion unit is constructed and arranged to translate the fiber assembly, linkage and optical element along a translational axis relative to the base.

In some embodiments, the system further comprising a probe connector that couples the proximal end of the fiber assembly and the proximal end of the linkage to the motion unit.

In some embodiments, the motion unit comprises: a rotary motor having a hollow shaft, wherein the probe connector is positioned in the hollow shaft, and wherein the hollow shaft is driven by the motion unit to rotate the linkage about the fiber assembly.

In some embodiments, the motion unit further comprises a linear motor that translates the fiber assembly and the linkage in a linear direction along the longitudinal axis.

In some embodiments, the linear motor further translates the rotary motor in the linear direction.

In some embodiments, the rotary motor and the linear motor operate independently of each other.

In some embodiments, the probe connector comprises a first portion coupled to the proximal end of the fiber assembly and a second portion coupled to the proximal end of the linkage, wherein the first portion is coupled to a first portion of the rotary motor that is rotationally fixed relative to the base, and wherein the second portion is coupled to a second portion of the rotary motor that rotates.

In some embodiments, the probe connector further comprises a bearing coupled between the first and second portions.

In some embodiments, the bearing comprises first and second bearings that are spaced apart from each other in the longitudinal direction.

In some embodiments, the bearing comprises at least one of a raised ring, a ball bearing, a radial ball bearing, or a thrust ball bearing.

In some embodiments, the linkage includes a flared end that prevents the bearing from sliding linearly along the linkage.

In some embodiments, a proximal end of the first portion of the probe connector includes a conical ferrule, wherein a proximal end of the fiber assembly is positioned at the conical ferrule, and wherein a proximal end of the hollow shaft of the rotary motor mates with the conical ferrule of the probe connector.

In some embodiments, the system further comprises an optical element adjacent the rotary motor, wherein the conical ferrule is positioned in the hollow shaft such that the proximal end of the fiber assembly is aligned with the optical element along the longitudinal axis.

In some embodiments, the conical ferrule of the probe connector is conformably positioned in a conical cavity of the hollow shaft of the rotary motor.

In some embodiments, the fiber assembly collects infrared energy from a body lumen tissue surface while the rotary motor of the motion unit rotates the linkage about the fiber assembly.

In some embodiments, the fiber assembly collects infrared energy from a body lumen tissue surface while the motion unit further translates fiber assembly along the longitudinal axis.

In some embodiments, the system further comprises a controller that processes the Infrared energy collected by the fiber assembly, and generates an output that includes temperature data related to the processed Infrared energy.

In some embodiments, the output includes at least one of a two dimensional (2D) graphical temperature map, a 1 dimensional (1D) graphical temperature map, a temperature value, an alarm, and a temperature rate of change.

In some embodiments, the controller performs the following steps to compensate for variability in rotational speed in the rotary motor: generate a two-dimensional array of the temperature data, the two dimensional array representing horizontal scan regions over a vertical scan region; identify a hotspot region, or other region of interest such as a hot or cold region, or a region that is most rapidly changing temperature the fastest in time or space, in the two-dimensional array of temperature data; performing a cross-correlation computation of neighboring horizontal scan regions; and performing an alignment computation to align the neighboring horizontal scan regions so that the hotspot region is aligned in the two-dimensional array of temperature data.

In some embodiments, the controller further displays the two-dimensional array of temperature data as a two-dimensional temperature map.

In some embodiments, the system further comprises a sheath surrounding the fiber assembly, linkage and optical element, wherein the linkage and optical element rotates relative to the sheath, and wherein the linkage, optical element and fiber assembly translates relative to the sheath.

In some embodiments, a distal end of the sheath includes a low-density polyethylene (LDPE) window segment within which the optical element receives the incident infrared energy.

In some embodiments, the system further comprises a proximal marker band and a distal marker band spaced apart from each other at the LDPE window segment.

In some embodiments, an outermost end of the sheath comprises a linear LDPE material.

In some embodiments, an outermost end of the sheath comprises at least one of a flexible ethylene co-polymer material or EVA material.

In some embodiments, an outermost end of the sheath comprises a coextrusion of Pebax over LDPE material.

In some embodiments, an outermost end of the sheath comprises a Pebax material that is bonded to the LDPE window by an adhesive-lined segment.

In some embodiments, the adhesive-lined segment includes Pebax.

In some embodiments, the outermost end of the sheath comprises a tip of reduced diameter relative to a diameter of the window region.

In some embodiments, the reduced-diameter tip is tapered or curved in shape.

In some embodiments, the reduced diameter tip comprises a flexible EVA copolymer.

In some embodiments, the outermost end is tapered or curved in shape.

In some embodiments, the outermost end comprises a Pebax segment coupled to the window region by a mechanical joint.

In some embodiments, the mechanical joint includes a perforation.

In some embodiments, the mechanical joint comprises heat fusing the Pebax segment to the window region at a spiral cut end of the window region.

In some embodiments, the mechanical joint comprises a metal band that is thermally bonded between the Pebax segment and the window region.

In some embodiments, the outermost end comprises an LLDPE segment coupled with the window region and wherein the mechanical joint comprises a metal band that is thermally bonded between the Pebax segment and the LLDPE segment.

In some embodiments, the distal end of the sheath includes a reinforcement unit that mitigates kinking of the distal end.

In some embodiments, the reinforcement unit comprises a lining within the distal end of the sheath.

In some embodiments, the lining comprises an ethylene vinyl acetate material.

In some embodiments, the reinforcement unit further comprises an insert comprising at least one of one or more balls, one or more pins, or a coiled material.

In some embodiments, the lining includes a neck for retaining the insert at a fixed location.

In some embodiments, the distal portion of the optical element includes an extension that mechanically communicates with the reinforcement unit.

In some embodiments, the system further comprises at least one marker band positioned at a distal end of the sheath, wherein the distal end of the fiber assembly is constructed and arranged to translate relative to the at least one marker band.

In some embodiments, the at least one marker band comprises a distal band and a proximal band, and wherein the first fiber assembly is constructed and arranged to translate between the distal band and the proximal band.

In some embodiments, the at least one marker band is constructed and arranged to cause a sensor in communication with a proximal end of the fiber assembly to produce a predetermined signal when the distal end of the at least one fiber receives infrared light from the at least one marker band.

In some embodiments, the at least one marker band is ring-shaped, and wherein a first portion of the ring has a first emissivity and wherein a second potion of the ring has a second emissivity.

In some embodiments, the first portion comprises a different material than the second portion.

In some embodiments, the first portion comprises a different color than the second portion.

In some embodiments, the first portion and the second portion comprise interior regions of the ring.

In some embodiments, the system further comprises a third portion of a third emissivity.

In some embodiments, the system further comprises a sensor assembly having a detector that receives the infrared energy from the fiber assembly, and converts the received infrared energy into temperature information signals.

In some embodiments, the sensor assembly is positioned at a positioning plate for aligning the sensor assembly with a proximal end of the fiber assembly.

In some embodiments, the positioning plate comprises an x-y-z positioning plate for adjusting the sensor assembly in at least one of an x, y, and z direction relative to the proximal end of the at fiber assembly.

In some embodiments, the sensor assembly comprises a cooling assembly constructed and arranged to cool one or more portions of the sensor.

In some embodiments, the system further comprises a controller that processes the infrared energy received by the sensor assembly and generates an output that includes temperature data related to the processed infrared energy.

In some embodiments, the sensor assembly includes an integrated housing in which a focusing lens, a cold diaphragm, and an immersion lens are affixed and separated by a predetermined distance.

In some embodiments, the fiber assembly is passive, and is constructed and arranged to only collect infrared energy from the tissue surface.

In another aspect, provided is a method for performing a medical procedure using the surgical instrument referred to herein.

### BRIEF DESCRIPTION OF THE DRAWINGS

The accompanying drawings, which are incorporated in and constitute a part of this specification, illustrate various embodiments of the present inventive concepts, and together with the description, serve to explain the principles of the inventive concepts. In the drawings:

FIG. 1 is a schematic view of a temperature mapping system including a temperature measurement probe, consistent with the present inventive concepts.

FIG. 2 is a magnified sectional side view of the distal portion of the temperature measurement probe of FIG. 1, consistent with the present inventive concepts.

FIGS. 3A, 3B, and 3C are perspective, schematic views of various optical elements in accordance with the present inventive concepts,

FIG. 4A is a cutaway perspective view of a distal portion of a temperature measurement probe, consistent with the present inventive concepts.

FIG. 4B is a cross-sectional view of a rotating assembly portion of the distal portion of the temperature measurement probe of FIG. 4A.

FIG. 4C is a cross-sectional view of a stationary assembly portion of the distal portion of the temperature measurement probe of FIG. 4A.

FIG. 5 is a cross-sectional view of a constrained distal assembly, consistent with the present inventive concepts.

FIG. 6 is a cross-sectional view of a proximal portion of a temperature measurement probe, consistent with the present inventive concepts.

FIG. 7 is a cross-sectional view of a proximal portion of a temperature measurement probe, consistent with other present inventive concepts.

FIG. 8A is a cross-sectional view of a proximal portion of a temperature measurement probe, consistent with the present inventive concepts.

FIG. 8B is an enlarged view of a region of the probe of FIG. 8A.

FIG. 9 is a cross-sectional view of a proximal portion of a temperature measurement probe, consistent with the present inventive concepts.

FIG. 10 is a cross-sectional view of a proximal portion of a temperature measurement probe, consistent with the present inventive concepts.

FIG. 11 is a cross-sectional view of a proximal portion of a temperature measurement probe, consistent with the present inventive concepts.

FIG. 12 is a cross-sectional view of a proximal portion of a temperature measurement probe, consistent with the present inventive concepts.

FIG. 13 is a view of a proximal portion of a temperature measurement probe, consistent with the present inventive concepts.

FIG. 14 is a cross-sectional view of a proximal portion of a temperature measurement probe, consistent with the present inventive concepts.

FIG. 15A is a perspective view of an optic sleeve, consistent with the present inventive concepts.

FIG. 15B is a cross-sectional side view of the optic sleeve of FIG. 15A.

FIGS. 16A-16C are views illustrating a method for enclosing a distal optic in a molded sleeve, consistent with the present inventive concepts.

FIGS. 17 and 18 are views of a method for coupling a fiber sheath and a distal ferrule of a temperature measurement probe, consistent with the present inventive concepts.

FIG. 19 is a cross-sectional view of a distal portion of a temperature measurement probe, consistent with the present inventive concepts.

FIG. 20 is a cross-sectional view of a distal optic sleeve at a portion of a temperature measurement probe, consistent with the present inventive concepts.

FIGS. 21A-H are cross-sectional views of various radiopaque sheath tips, consistent with the present inventive concepts.

FIG. 22 is a cross-sectional view of a non-kinking sheath tip, consistent with the present inventive concepts.

FIG. 23 is a cross-sectional view of another non-kinking sheath tip, consistent with the present inventive concepts.

FIG. 24 is a perspective view of a probe configured to include a multi-toned marker band about its sheath, consistent with the present inventive concepts.

FIG. 25 is an image of a scan result illustrating a misaligned hot spot, which is addressed by a temperature measurement probe, consistent with some present inventive concepts.

FIG. 26 is a method for realigning A-scans of a hot spot region, consistent with some present inventive concepts.

FIGS. 27A-27O are views of embodiments of different configurations of a distal end of a probe, consistent with some present inventive concepts.

FIG. 28 is a view of a proximal region of a temperature mapping system of FIGS. 1 and 6-11, consistent with some present inventive concepts.

FIG. 29 is a view of a proximal region of another embodiment of a sensor assembly in communication with a focusing lens at a proximal region of a temperature mapping system, consistent with some present inventive concepts.

### DETAILED DESCRIPTION

Reference will now be made in detail to the present embodiments of the inventive concepts, examples of which are illustrated in the accompanying drawings. Wherever possible, the same reference numbers will be used throughout the drawings to refer to the same or like parts.

The terminology used herein is for the purpose of describing particular embodiments and is not intended to be limiting of the inventive concepts. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element or intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

For example, it will be appreciated that all features set out in any of the claims (whether independent or dependent) can be combined in any given way.

Provided herein is a temperature measurement system for producing a temperature map for multiple locations, such as a two or three dimensional surface of a patient's tissue. The system can include one or more sensors, such as infrared (IR) light detectors or other infrared sensors. In other embodiments, the system can include thermistor or thermocouple sensors. The system can include a reusable portion, and one or more disposable portions. The system can include a probe, such as a probe constructed and arranged to be inserted into a body lumen such as the esophagus, respiratory tract, or colon. Probe can include an elongate member such as a shaft, and the system can be constructed and arranged to measure temperature at multiple tissue locations positioned at the side of the elongate member and/or forward of the distal end of the elongate member. The system or probe can be constructed and arranged as described in applicant's co-pending International Patent Application Serial Number PCT/US2011/061802 filed Nov. 22, 2011, PCT/US13/76961 filed Dec. 20, 2013, or PCT/US15/33680 filed Jun. 2, 2015, the content of each of which is incorporated by reference in its entirety above.

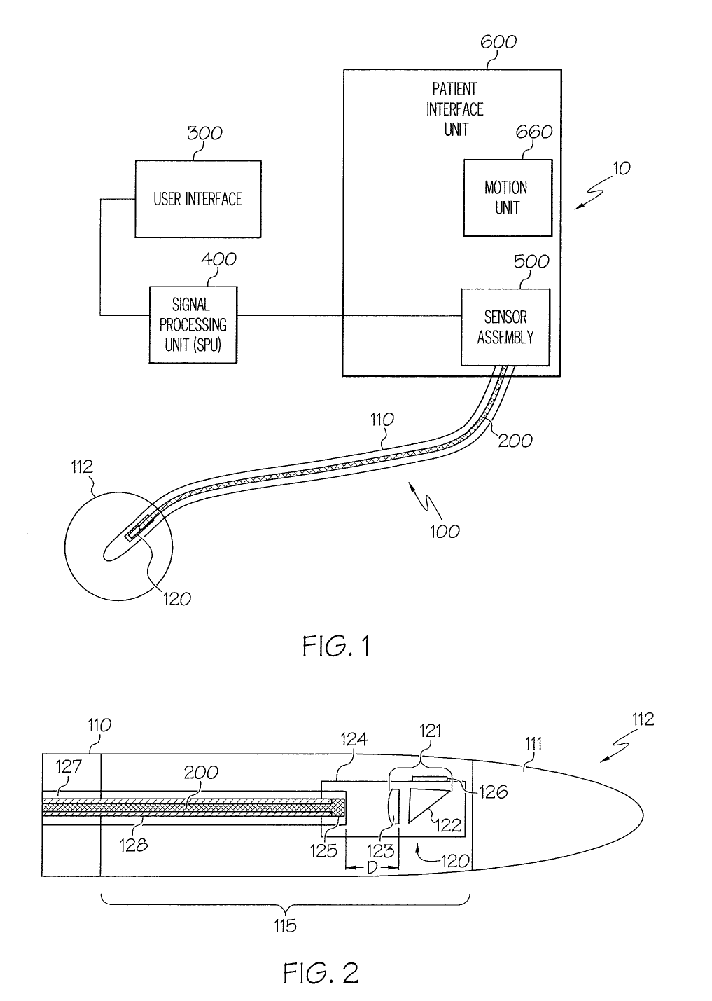

Referring now to FIG. 1, a schematic view of a temperature mapping system 10 including a temperature measurement probe is illustrated, consistent with the present inventive concepts. System 10 includes probe assembly 100, sensor assembly 500, fiber assembly 200, user interface 300, signal processing unit (SPU) 400, and patient interface unit 600.

Probe assembly 100 includes shaft 110 which slidingly receives fiber assembly 200, which includes one or more elongate filaments, or fibers. The fiber or fibers can comprise one or more materials highly transparent to one or more ranges of infrared light wavelengths, such as one or more materials selected from the group consisting of: zinc selenide; germanium; germanium oxide; silver halide; chalcogenide; a hollow core fiber material; and combinations of these. The fibers can be configured to be highly transmissive with respect to infrared light with wavelengths between 6 μm to 15 μm, or between 8 μm and 11 μm. In some embodiments, fiber assembly 200 comprises multiple fibers, such as multiple fibers in a coherent or non-coherent bundle.

In some embodiments, the probe assembly 100 includes an optical assembly 120 positioned at a distal end of the fiber assembly 200 thereof. The optical assembly 120 and the fiber assembly 200 may be constructed and arranged to collect electromagnetic energy at wavelengths at least in the infrared light range emanating from one or more surface locations (e.g. one or more tissue surface locations) positioned radially out from the central axis of the distal portion of shaft 110. The collected infrared light travels proximally within fiber assembly 200 and is received by sensor assembly 500. Sensor assembly 500 converts the received infrared light to one or more information signals that are transmitted to SPU 400.

In some embodiments, patient interface unit 600 includes motion unit 660 that causes an optical assembly 120 positioned at a distal end 112 of probe assembly 100 to rotate relative to the fiber assembly 200. In some embodiments, motion unit 660 is coupled to the optical assembly 120 via a linkage 127 (see FIG. 2, for example). In some embodiments, the motion unit 660 operates to rotate the linkage 127 to cause the optical assembly 120 to rotate relative to the fiber assembly 200. In some embodiments, the linkage 127 is elongated and includes a channel through which the fiber assembly 200 passes. In such an embodiment, the motion unit 660 causes the linkage 127 to rotate about the fiber assembly 200, and causes the optical assembly 120 to rotate relative to the fiber assembly 200. In embodiments, the fiber assembly 200 can be considered to be rotationally fixed, while the linkage 127 and the optical assembly 120 coupled thereto rotate relative to the fixed fiber assembly 200.

In some embodiments, the motion unit 660 further causes the fiber assembly 200, and linkage 127 and optical assembly 120, to translate, or induce linear motion, relative to probe shaft 110, such as to collect infrared light from a series of tissue locations (e.g. a contiguous or discontiguous surface of tissue). The linkage 127, also referred to herein for the purpose of discussion as a “torque coil”, may surround fiber assembly 200 along some or all of the length of the fiber assembly 200. Torque coil 127 is configured to transmit rotational forces from motion unit 660 from a proximal portion of fiber assembly 200 in communication with sensor assembly 500, to an IR collection region of the optical assembly 120 at the distal end of fiber assembly 200, such that elements of the collection region, in particular, an optical mirror, rotates within the shaft 110 as described herein. In some embodiments, torque coil 127 comprises an elongated, flexible tube-shaped body having a central channel, the body comprising a woven fabric of multiple wires or other filaments such as stainless steel or titanium wires. In some embodiments, the torque coil 127, or linkage, comprises an elongated tubular material that is torsionally rigid and longitudinally flexible. In some embodiments, torque coil 127 comprises a single-layer or multiple-layer spring. In some embodiments, the spring may comprise rounded or flat wires. In some embodiments, the spring comprises at least one of wire, metal, alloy, steel, graphite, composite, plastic, or other suitable material. Although the linkage 127 is described herein as a “torque coil”, embodiments of the present inventive concepts are not limited thereto, and other types of suitable rotational linkages may be employed for this purpose. In some embodiments, laser-cut tubing can be employed as the linkage.

In some embodiments, referring now to FIGS. 1 and 2, a slip ring 128, a bearing, a lubricious sleeve, or the like can be positioned between fiber 200 and torque coil 127, e.g., positioned in a channel or lumen of the torque coil 127 through which fiber assembly 200 also extends, so that the torque coil 127 can rotate freely about fiber assembly 200 in a substantially unrestrained and continuous or intermittent manner.

SPU 400 converts the one or more information signals received from sensor assembly 500 into a series of temperature measurements that can be correlated to the series of tissue locations, such as to provide information regarding temperatures (e.g. average temperatures) present on a two and/or three dimensional tissue surface. The information provided by sensor assembly 500 is used by SPU 400 to produce a table of collection location measured temperatures, which represent an estimated, averaged temperature for the collection location, as described above. The table provided by SPU 400 can be represented (e.g. by user interface 300) in the form of a temperature map or other display of data correlating to the geometry of the multiple collection locations. In some embodiments, the multiple collection locations comprise a segment of tubular tissue, such as a segment of esophagus, and the temperature map is a two dimensional representation of the “unfolded” luminal wall or other body tissue. In other embodiments, a three dimensional representation of the luminal wall or other body tissue can be provided. The table or other representation can be updated on a regular basis.

Continuing to refer to FIGS. 1 and 2, distal end 112 of the probe shaft 110 can comprise a rounded tip, or sheath 111, and/or relatively infrared transparent tube (i.e. an infrared transmissive tube) configured for atraumatic insertion of probe 100 into a body lumen of a patient. In some embodiments, sheath 111 is part of the shaft 110, and extends from the proximal end to the distal end 112 of probe 100. In other embodiments, sheath 111 extends along at least of a portion of the shaft 110. In other embodiments, sheath 111 is formed separately from the shaft 110 and coupled (e.g., glued, bonded, or the like) to the distal end 112 of the shaft 110, thereby forming part of the distal end 112 of the shaft 110. In various embodiments, shaft 110 can comprise a material selected from the group consisting of: polyethylene; polyimide; polyurethane; polyether block amide; and combinations of these. Shaft 110 can comprise a braided shaft and/or include one or more braided portions constructed and arranged to provide increased column strength and/or improve response to a torque applied at or near proximal end 111 of shaft 110. Probe 100 can be configured for insertion over a guidewire, not shown, but typically where shaft 110 includes a guidewire lumen or distal guidewire sidecar as is known to those of skill in the art.

Distal portion 112 of shaft 110 may include a relatively infrared transparent tube (i.e. an infrared transmissive tube) or window 115 comprising a tubular segment, which can include at least a portion which is transparent to, or relatively transparent to, infrared light. In some embodiments, window 115 is part of the sheath 111, or an opening in the sheath 111. In some embodiments, window 115 can comprise a material selected from the group consisting of: polyethylene such as high density polyethylene (HDPE) or low density polyethylene (LDPE); germanium or similarly infrared transparent materials; and combinations of these. In embodiments where shaft 110 includes a braid or other reinforcing structure, window 115 or a portion of window 115 can be void of the reinforcing structure so as to be transmissive of the infrared light energy desired for collection.

Shaft 110 can be rigid, flexible, or can include both rigid and flexible segments along its length. Fiber assembly 200 can be rigid, flexible, or can include both rigid and flexible segments along its length. Shaft 110 and fiber assembly 200 can be constructed to be positioned in a straight or curvilinear geometry, such as a curvilinear geometry including one or more bends with radii less than or equal to 4 inches, less than or equal to 2 inches, or less than or equal to 1 inch, such as to allow insertion into the esophagus via a nasal passageway. In some embodiments, shaft 110 and fiber assembly 200 comprise sufficient flexibility along one or more portions of their length to allow insertion of probe 100 into a body lumen or other body location, such as into the esophagus via the mouth or a nostril, the respiratory tract via the mouth or a nostril/nasal cavity, or into the lower gastrointestinal tract via the anus, and/or into the urethra. Shaft 110 can comprise an outer diameter less than 15 Fr, such as a shaft with a diameter less than 12 Fr, less than 9 Fr, or less than 6 Fr.

In some embodiments, portions of the fibers of the fiber assembly 200 comprise a surface with a coating, such as an anti-reflective (AR) coating. System can include one or more components that include an optical surface that receives infrared light and/or from which infrared light is emitted. These optical surfaces can include one or more anti-reflective coatings, such as a coating selected from the group consisting of: a broadband anti-reflective coating such as a coating covering a range of 6 μm-15 μm or a range of 8 μm-11 μm; a narrow band anti-reflective coating such as a coating covering a range of 7.5 μm-8 μm or a range of 8 μm-9 μm; a single line anti-reflective coating such as a coating designed to optimally reflect a single wavelength or a very narrow range of wavelengths in the infrared region; and combinations of these. Anti-reflective coatings can be included to improve transmission by up to 30% per surface by reducing Fresnel losses at each surface. Anti-reflective coatings can be constructed and arranged to accept a small or large range of input angles.

In some embodiments, fiber assembly 200 comprises a cladding to cause and/or maintain total internal reflection of the infrared light as it travels from the distal to proximal end of fiber assembly 200. Alternatively or additionally, fiber assembly 200 can comprise a coil, braid or other twist resisting structure surrounding one or more optical fibers.

Referring again to FIG. 2, distal end 112 of probe 100 can include an optical assembly 120 comprising an optical element 121 and a holder 124 that are aligned or otherwise extend along a common longitudinal axis as the fiber assembly 200. Components of optical assembly 120 can include similar or dissimilar materials to the materials of optical fibers of the fiber assembly 200, such as materials configured to pass (e.g. be relatively transparent to) infrared light in the 6-15 micron wavelength range, such as light in the 8-11 micron wavelength range, as has been described herein. Elements of fiber assembly 200 having an optical surface, such as a distal end of fibers of the fiber assembly 200, can include an anti-reflective coating.

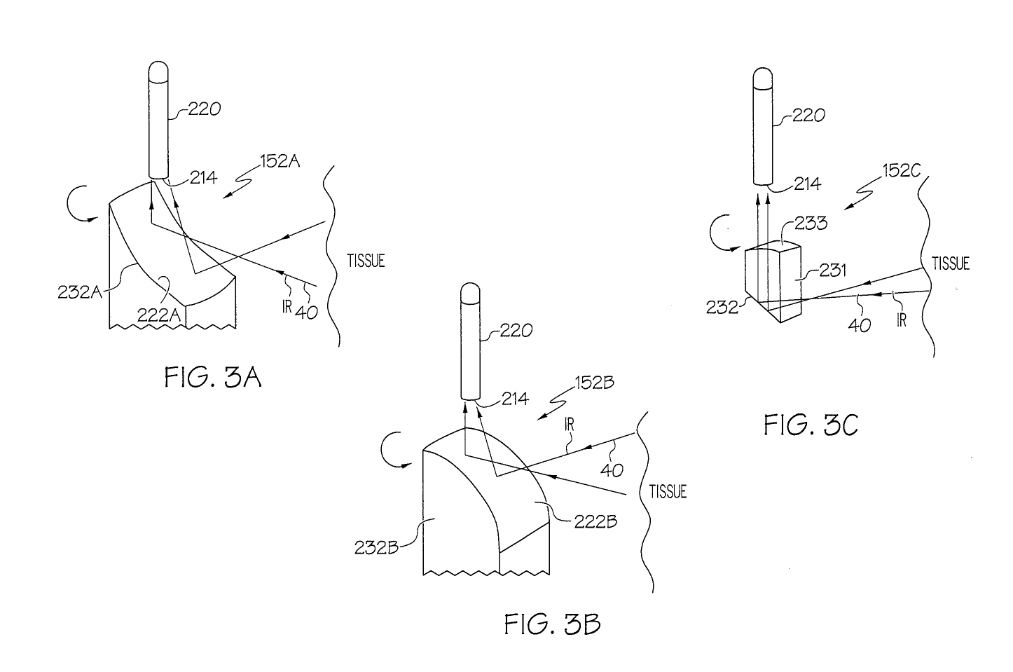

In some embodiments, optical element 121 includes a mirror 122 and a focusing lens 123 positioned in holder 124. In some embodiments, mirror 122 and focusing lens 123 are distinct structural elements and separate from each other by a predetermined distance. In other embodiments, as shown in FIGS. 3A-3C, a mirror and focusing lens are integrated, unitary, or otherwise part of the same structural element, for example, a reflective or refractive element.

Optical element 121 can otherwise include one or more optical components used to perform an action on collected infrared light, such as an action selected from the group consisting of: focus; split; filter; transmit without filtering (e.g. pass through); amplify; refract; reflect; polarize; and combinations of these. To achieve this, holder 124 can include one or more optical components selected from the group consisting of: optical fiber; lens; mirror; filter; prism; amplifier; refractor; splitter; polarizer; aperture; optical frequency multiplier and combinations of these. Holder 124 can include a window or opening 126 that is aligned with mirror 122 for receiving IR signals from a surface of a tissue area. In some embodiments, window 126 can be constructed and arranged to permit the transmission of IR signals with little or no impact on the received IR signals. In doing so, in some embodiments, window 126 may have different transmissivity characteristics than holder body 124. For example, window 126 may be transparent with respect to IR light. In other embodiments, window 126 may have same or similar transmissivity characteristics as the holder body 124.

Holder 124 can be coupled to a distal end of torque coil 127, which, in turn, extends about fiber assembly 200. In some embodiments, torque coil 127 can be driven by motion unit 660 to rotate about fiber assembly 200. In doing so, torque coil 127 causes holder 124 and its corresponding optics 121 including mirror 122, or including mirror 122 and lens 123, to likewise rotate. In some embodiments, as shown in FIG. 2, optical element 121 including both mirror 122 and focusing lens 123 rotate with holder 124 during a temperature measurement operation.

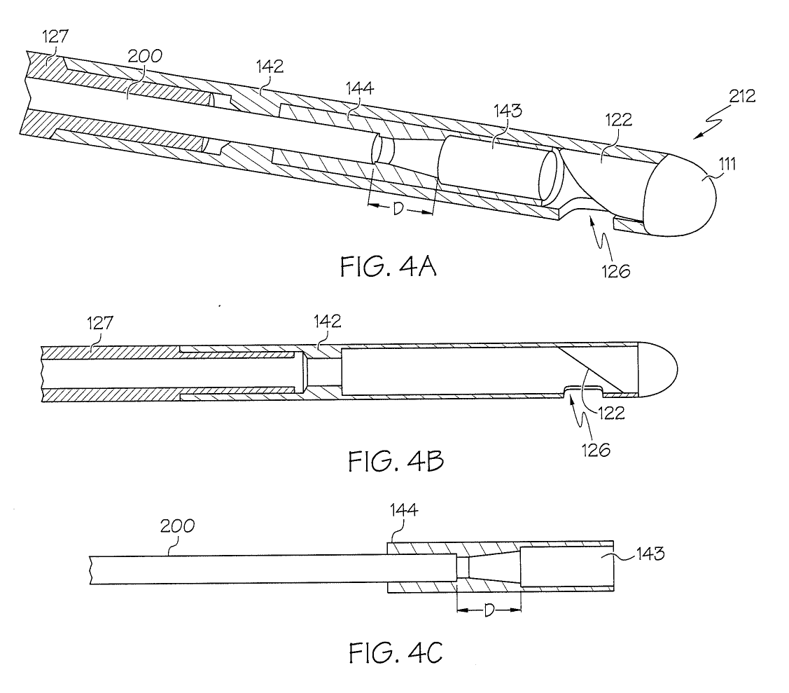

In other embodiments, for example, described in detail below with respect to FIGS. 4A-4C, a dual-holder configuration is provided, whereby an inner holder 144 is attached to a lens 143, which is held in a stationary position relative to the fiber assembly 200, the lens 143 in turn being held in a stationary position relative to a mirror 122 which mirror is rotated by an outer holder 142 connected to the linkage 127. In some embodiments, lens 143 is directly affixed to the distal end of fiber assembly 200 or affixed to inner holder 144 and does not rotate, whereby mirror 122, inner holder 144 (see FIG. 4C), and torque coil 127 may rotate relative to fiber assembly 200 and lens 143.

During a temperature measurement operation, IR light which is emitted from a particular tissue location proximate to the distal portion of fiber assembly 200, may then pass through sheath 111, where it is redirected by optical element 121 toward the distal end of fiber assembly 200. For example, referring again to FIG. 2, IR light collected from a surface of a tissue area is directed by mirror 122 to focusing lens 123, which is configured to focus the IR light toward the fiber assembly 200. The redirected light is passively transmitted from the distal end up the passive fiber assembly 200 to its proximal end, where a sensor, or more specifically, a proximal lens, receives and focuses the energy onto the sensor and signal processing unit 400 perform calculations on the received and collected IR energy. A number of different readings and determinations can be performed by the signal processing unit 400. For example, average temperature can be calculated for the tissue area based on the amount of IR light which has been collected. In applications where the average temperature is to be displayed, or otherwise presented as a temperature versus two-dimensional location map (i.e. a map of multiple tissue locations), the area of each projection of optical assembly 120 is used to create the temperature map and can be known or otherwise estimated.

Referring again to FIG. 1, proximal end of fiber assembly 200 is in optical communication with sensor assembly 500 such that the collected light is received by sensor assembly 500. In some embodiments, a signal produced by sensor assembly 500 based on the collected light is correlated by SPU 400 to an estimated, average temperature, hereinafter “measured temperature”, for that particular tissue location, hereinafter the “collection location”. This measured temperature represents an average temperature of the entire surface of the collection location, which can include multiple different temperatures across its entire surface. In other words, the collected infrared light from each collection location travels proximally through fiber 200 as a single, undividable signal correlating to an average temperature of the entire collection location. Errors in the measured temperature can be caused by a factor selected from the group consisting of: unaccounted for and/or unknown infrared signal losses along an optical pathway of the system 10; unaccounted for and/or unknown infrared signal gains (e.g. an extraneous input of infrared light) along optical pathway; sensor assembly 500 inaccuracies or spurious signals; electrical signal noise; and combinations of these.

As described herein, motion unit 660 can cause fiber assembly 200, and the linkage 127 and optical assembly 120 to translate, or be moved in a linear direction, relative to probe shaft 110, or sheath 111. In some embodiments, the motion unit 660 can cause the optical assembly 120 at a distal end 112 of the probe 100 to rotate relative to the fiber assembly 200, and can cause the linkage 127 to rotate about the fiber assembly 200. To achieve this, motion unit 660 can include a rotary motor and/or linear translation motor assembly, respectively. In some embodiments, sensor assembly 500 and a rotary motor of the motion unit 660 can be positioned on a translation table, which in turn can be moved linearly by linear translation motor assembly, for example, as described in PCT/US15/33680 filed Jun. 2, 2015, incorporated by reference herein.

The translation or linear motion of the fiber assembly 200 and optical assembly 120 at the distal end 112 can be achieved by linear translating assembly of the motion unit 660, which applies an axial force to cause torque coil 127, fiber assembly 200, and optical assembly 120 to move forward and back within shaft 110, and in particular, relative to sheath 111. In some embodiments, the magnitude of reciprocating motion by the linear translating assembly is constructed and arranged to collect temperature information from a sufficient length of the esophagus during a cardiac ablation procedure.

The rotating motion of the optical assembly 120 about the fiber assembly 200 can be achieved by rotary motor of the motion unit 660, such as one or more continuous 360° rotations or partial circumferential rotation (e.g. 45° to 320° reciprocating rotation).

User interface 300 can include a monitor or the like which can comprise at least one touch-screen or other visual display monitor. User interface 300 can be stored in memory and executed by a computer processor. User interface 300 can optionally further include an input device, which can include a component configured to allow an operator of system 10 to enter commands or other information into system 10, such as an input device selected from the group consisting of: monitor such as when monitor is a touch screen monitor; a keyboard; a mouse; a joystick; and combinations of these. In some embodiments, command signals provided by user interface 300, such as via input device, can be transmitted to SPU 400 via a conductor. Accordingly, user interface 300 can present temperature information, for example, displayed as a temperature map, temperature values, present temperature information, past temperature information, and so on, in response to IR energy received at a body lumen wall or related tissue surface from probe assembly 100.

FIGS. 3A, 3B, and 3C are perspective, schematic views of various optical elements in accordance with the present inventive concepts, for example those described in connection with optical element 121 of FIG. 2. In the embodiments of FIGS. 3A and 3B a reflective optical element 152A, 152B, including a mirrored surface 232A, 232B, is provided. In each example of FIGS. 3A and 3B, the mirrored surface is non-planar, so as to include an integrated lens effect. In the embodiment of FIG. 3A, the mirrored surface 232A is concave in profile 222A, and in the embodiment of FIG. 3B, the mirrored surface 232B is concave in profile 222B. In either case, the non-planar profile of the mirrored surface 222A, 222B operates to provide a reflection of the incident IR radiation, redirecting the IR radiation in a direction toward the distal end of the fiber assembly and further operates to provide a focusing of the re-directed IR radiation, depending on the optical parameters of the non-planar profile. The presence of mirrors in the configurations of FIGS. 3A and 3B eliminate the need for expensive IR materials.

In the embodiment of FIG. 3C, a refractive optical element 152C is illustrated in which incident IR radiation enters the body of the optical element 232 at an incident surface 231. Accordingly, optical element 152C is formed of a material that is transmissive to IR light. IR transmissive materials may include, for example, germanium, zinc selenide, or related material. In some embodiments, the incident surface 231 is planar as shown; however embodiments of the present inventive concepts are not limited thereto, and the incident surface can be non-planar such as convex, concave, or textured in profile so as to provide a focusing function. Refractive optical element 152C can further include an internally reflective mirror portion, for example, similar to mirror 122 of FIG. 2, and a focal lens portion. Accordingly, optical element 152C in the present embodiment includes an optical refractor that includes planar surface 231, angled surface 232, and contoured surface 233.

The optical element 152C includes planar surface 231, angled surface 232, and/or contoured surface 233 can comprise a flat, convex, concave, curved, and/or an irregularly shaped surface configured to collect IR light 40 emitted from a surface of tissue area. In various embodiments, planar surface 231 and/or contoured surface 233 can include an anti-reflective coating to accommodate efficient transfer of incident IR radiation. In some embodiments, as shown, contoured surface 233 of refractive optical element 152C functions as a focusing lens, and in doing so, may comprise a convex geometry, or alternatively, a concave, curved, or irregularly shaped geometry.

Continuing to refer to FIG. 3C, IR light 40 emitted from the tissue area is collected by optical element 152C at surface 231, and travels through optical element 152C toward angled surface 232. Angled surface 232 can be at an angle of 45° relative to the axis of rotation, and can be coated, for example with a reflective coating such as a protected aluminum (PAL) or gold coating. Angled surface 232 can be configured to reflect IR light 40 in a direction toward convex surface 233 of optical element 152C. In some embodiments, angled surface 232 can comprise an angle greater than or less than 45°. In some embodiments, the incident surface 231 is planar as shown; however embodiments of the present inventive concepts are not limited thereto, and the incident surface 231 can be non-planar such as convex, concave, or textured in profile so as to perform a focusing function.

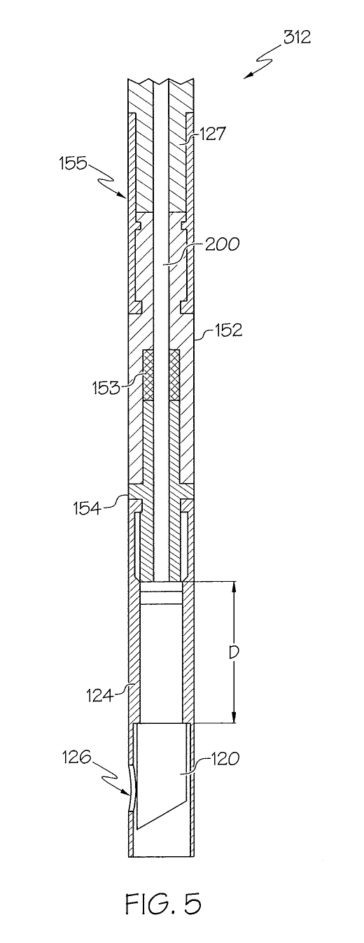

As described herein, motion unit 660 may include a motor that provides linear motion of the fiber assembly 200 and optical assembly 120 at the distal region 112. In some embodiments, the distal end or ends 214 of the fiber assembly 200 is separated from focusing lens 123 by a physical gap, distance D, referring again to FIG. 2. D can be varied, either during use or in a manufacturing process, such as to set the magnification of IR light throughout optical assembly 120. However, the reciprocating motion by the linear translating assembly can provide forces that separate the fiber assembly 200 from the optical element 121. In doing so, temperature measurements may become inaccurate if the predetermined distance D between the distal fiber tip and the focusing lens 123 is changed from a known distance D to a different distance. In order to maintain distance D, a bearing 125 or related element, for example, collar 153 shown in FIG. 5, can be coupled between the distal end of the fiber assembly 200 and the holder 124 to prevent sliding or undesirable motion of the fiber tip relative to the optical element 121 that would otherwise change distance the D. Accordingly, this configuration eliminates the variation in distal optic distance during operation, for example, when the probe 100 is engaged in linear travel, for example, back and forth motion.

Accordingly, a feature is that manufacturing processes do not significantly affect, or change, distance D between distal fiber tip of the fiber assembly 200 and focusing lens 123. In manufacturing, the system can be calibrated to account for the tolerances around distance D. The fiber assembly 200 and torque coil 127 may experience considerable compliance and stretching due to forces caused by translation, which can change the distance D. Those forces resulting in changes in distance D during translation or rotation may result in changes in the amount of energy that is collected by the fiber and therefore result in changes in temperature during the push and pull cycles of translating and rotating motion. Bearing 125 may maintain a preload on the fiber within the torque coil 127. The preload takes up the push/pull forces caused during translation and/or rotation and inhibit changes in distance D resulting in consistent temperature reading throughout the reciprocation cycle.

FIG. 4A is a cutaway perspective view of a distal portion 212 of a temperature measurement probe 100, consistent with the present inventive concepts. FIG. 4B is a cross-sectional view of a rotating assembly portion of the distal portion 212 of the temperature measurement probe 100 of FIG. 4A. FIG. 4C is a cross-sectional view of a stationary assembly portion of the distal portion 212 of the temperature measurement probe 100 of FIG. 4A.

Distal end 212 of probe 100 can be similar to distal end 112 described in FIG. 2, except that the distal end 212 of probe 100 in FIGS. 4A-4C includes first and second holders; namely, a dual-holder configuration including an inner holder 144 and an outer holder 142. In the present embodiment, the inner holder 144 is fixedly attached to a lens 143, which is held in a rotationally stationary position relative to a mirror 122 which is rotated by the outer holder 142.

More specifically, as shown in FIG. 4c, the fiber assembly 200 is affixed to the inner holder 144, also referred to as an optic holder, at which lens 143 or related optical element is positioned. Inner holder 144 is independent of the outer holder 142, which outer holder 142 is coupled to the torque coil 127 so that the outer holder 142 can move in a rotational motion independently of the rotationally fixed inner holder 144. In the present embodiment, as shown in FIG. 4A, the fiber assembly 200, inner holder 144, and lens 143 are rotationally stationary relative to the torque coil 127 and mirror 122, while the torque coil 127, outer holder 142, and mirror 122, which are together rotatable relative to the rotationally fixed fiber 200, inner holder 144 and lens 143. As shown in FIG. 4C, the inner holder 144 separates the fiber assembly 200 from the lens 143 by a predetermined distance D. Accordingly, in this configuration, optic holder 144 prevents a variation in distal optic distance D during operation, for example, when the probe 100 is engaged in linear travel, for example, back-and-forth motion.

FIG. 5 is a cross-sectional view of a constrained distal assembly 312, consistent with the present inventive concepts.

The distal assembly 312 can include optical assembly 120, holder 124, torque coil 127, fiber assembly 200, coupling 152, collar 153, distal ferrule 154, and distal termination 155.

As described herein, the fiber assembly 200 is preferably stationary, i.e., does not rotate, while the optical assembly 120 rotates relative to the stationary fiber assembly 200. The distal coupling 152 is coupled to the stationary fiber 200 between the distal ferrule 154 and distal termination 155. Torque coil 127 causes coupling 152, distal ferrule 154, and holder 124 to rotate, which in turn cause the optical assembly 120 to rotate.

A space or gap can extend between distal ferrule 154 and coupling 152. Collar 153 can be positioned in this space or gap. Collar 153 is affixed to the fiber assembly 200, for example, bonded to a Polyetheretherketone (Peek) sheath, or other plastic material surrounding the fibers of the fiber assembly 200. The collar 153 therefore allows for rotation of torque coil 127 about the fiber 200, while operating with distal ferrule 154 to prevent linear movement of the fiber 200 relative to torque coil 127, coupling 152, and distal ferrule, so that a distance D between distal end of fiber of the fiber assembly 200 and optical element 120 is maintained.

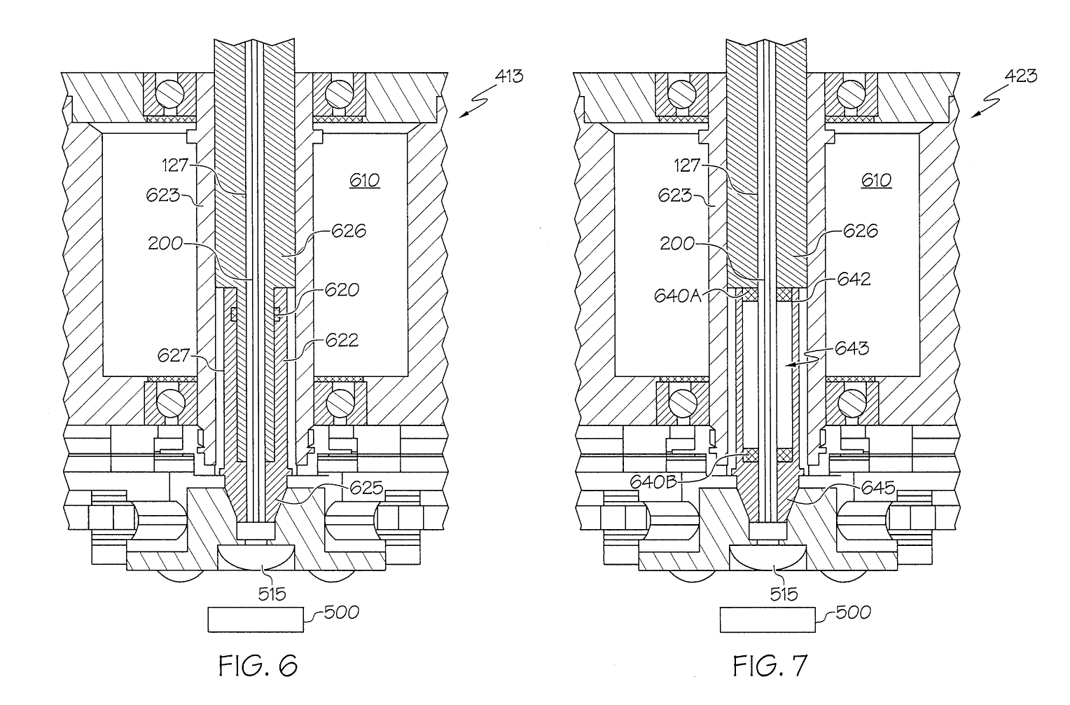

FIG. 6 is a cross-sectional view of a proximal portion 413 of a temperature measurement probe, consistent with the present inventive concepts. The temperature measurement probe may include components that are similar to or the probe 100 described herein, and descriptions thereof are not repeated due to brevity

As described above with respect to FIG. 1, motion unit 660 of patient interface unit 600 can include a rotary motor. FIG. 6 illustrates a rotary motor 610 that can be part of motion unit 660, and that rotates torque coil 127. Sensor assembly 500 and rotary motor 660 can translate in the linear direction along with a translation table (not shown), as driven by a linear translation motor assembly (not shown), for example, similar to a system described in PCT/US15/33680 filed Jun. 2, 2015, incorporated by reference above. In some embodiments, linear translation motor assembly of motion unit 660 moves torque coil 127 and fiber assembly 200 together in a linear direction.

In some embodiments, rotary motor assembly 610 includes a central hollow shaft 623 into which a probe connector 626 through which a proximal end of fiber assembly 200 extends. Rotary motor 610 can include a stator, rotor, and/or other well-known rotary motor components, which in turn can initiate a rotary motion in hollow shaft 623 which in turn rotates probe connector 626 positioned in shaft 623. Probe connector 626 can be removably attached to shaft 623, for example in a manner similar to embodiments described in PCT/US15/33680 filed Jun. 2, 2015, incorporated by reference herein.

A rotational encoder wheel (not shown) may be fixedly attached to an end of rotor shaft 623, which can be tapered, conical, circular, or other shape that provides benefits described herein. The encoder wheel provides feedback to the motor controller to precisely control the angular position, angular velocity, or angular acceleration of the rotor shaft 623 relative to the stator. In this manner, the rotation of the inserted probe connector 626 and, in turn, rotation of the corresponding fiber assembly 200, can be precisely controlled.

The end of rotor shaft 623 can be concave and conical or otherwise circular for receiving a mating nose of the probe assembly, for example, probe assembly 100 shown in FIG. 1. The conical or circular arrangement allows for reliable optical coupling between the proximal end of the fiber 200, at which the collected IR energy signals are output, with the optical element of the sensor 500, ensuring proper alignment and spacing therebetween. In alternative embodiments, other concave/convex nose shapes may be employed and are equally applicable to the principles of the inventive concepts. Such shapes can include but not be limited to parabolic, elliptical, semi-spherical, stepped, and the like

Positioned at a proximal end of shaft 623 may include a long proximal bushing 622 that includes a conical proximal ferrule 625. Proximal ferrule 625 is coupled to an outermost tip of fiber assembly 200 and holds the fiber assembly 200 in a rotationally stationary position relative to sensor assembly 500. Proximal lens 515 may focus light output from fibers of the fiber assembly 200 onto sensor assembly 500. A portion 627 of probe connector 626 extends through a hollow central region of bushing 622 and is positioned about fiber assembly 200, and is rotatable about the fiber assembly 200. This portion 627 of probe connector 626 is positioned at a hollow interior of stationary proximal ferrule 625 extending from stationary fiber bushing 622. In alternative embodiments, other concave/convex nose shapes may be employed and are equally applicable to the principles of the inventive concepts. Such shapes can include but not be limited to parabolic, elliptical, semi-spherical, stepped, and the like. In the conical embodiment depicted in FIG. 6, the conical feature ensures capture and seating of the probe in a repeatable, final position where the proximal end of the fiber can maintain concentricity with the proximal lens 515.

Proximal bushing 622 can include grooves, ridges, or the like, for example, similar to FIG. 10, that snap-fit together with a raised ring 620, ball bearing, or the like on the probe connector 626. The snap-fit configuration can include a mechanical interference that captures proximal ferrule 625 over raised ring 620. Proximal ferrule 625 can be formed of plastic PEEK or the like that provides sufficient compliance for fitting over raised ring 620. A tapered configuration may be presented to permit a press fit between proximal ferrule 625 and raised ring 620. There would also be some tapers to allow press fit. Raised ring 620 is positioned about fiber assembly 200 in the hollow center of proximal bushing 622. Raised ring 620 may include a ball bearing or the like that separates the rotational elements, in particular, probe connector 627 and torque coil 127, from stationary elements, in particular, proximal bushing 622.

FIG. 7 is a cross-sectional view of a proximal portion 423 of a temperature measurement probe, consistent with other present inventive concepts. The temperature measurement probe may include components that are similar to or the probe 100 described herein, and descriptions thereof are not repeated due to brevity.

Proximal portion 423 of a temperature measurement probe of FIG. 7 is different than that illustrated in FIG. 6 in that proximal portion 423 includes dual bearings 640A, B (generally, 640). A first bearing 640A is positioned at a distal end of ferrule 642 and pressed onto a surface of probe connector 626. Second bearing 640B is positioned at the conical proximal end of the ferrule 645. A gap 643 is present between the first bearing 640A, second bearing 640B, and a portion of torque coil 127 in ferrule 642. During operation, similar to the probe shown in FIG. 6, proximal ferrule 642 and fiber assembly 200 are stationary, while probe connector 626 and torque coil 127 rotate about fiber 200. The arrangement of the bearings 640A, 640B in this manner provide stability while operating at high rotational speeds.

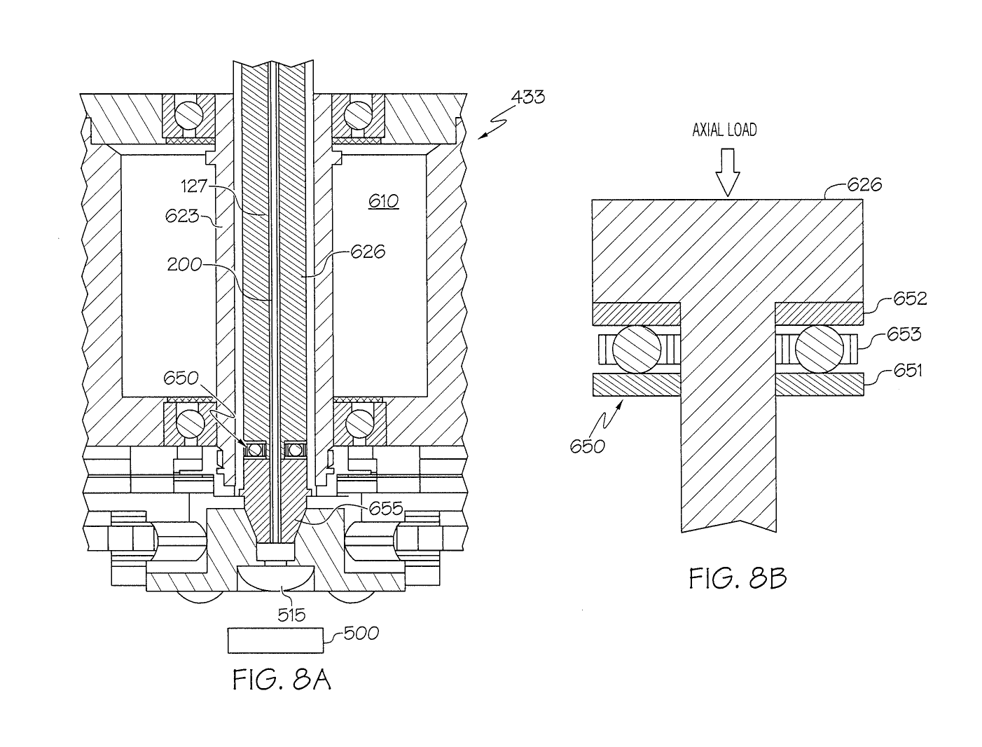

FIG. 8A is a cross-sectional view of a proximal portion 433 of a temperature measurement probe, consistent with the present inventive concepts. FIG. 8B is an enlarged view of a region of the probe of FIG. 8A.

The temperature measurement probe may include components that are similar to or the probe 100 described herein, and descriptions thereof are not repeated due to brevity.

Proximal portion 433 of a temperature measurement probe of FIGS. 8A and 8B is different than those illustrated in FIGS. 6 and 7 in that proximal portion 433 includes a single thrust ball bearing 650 between stationary proximal ferrule 655 and rotatable probe connector 626. Thrust ball bearing 650 can accommodate higher axial loads than a single radial ball bearing, shown in FIG. 8B. Here, first race 651 spins in relation to second race 652. In conventional axial loads, the radial ball bearing is loaded with a shallow contact angle across the balls. However, the thrust bearing 650 is loaded in a normal direction across the balls 653, accommodating high loads.

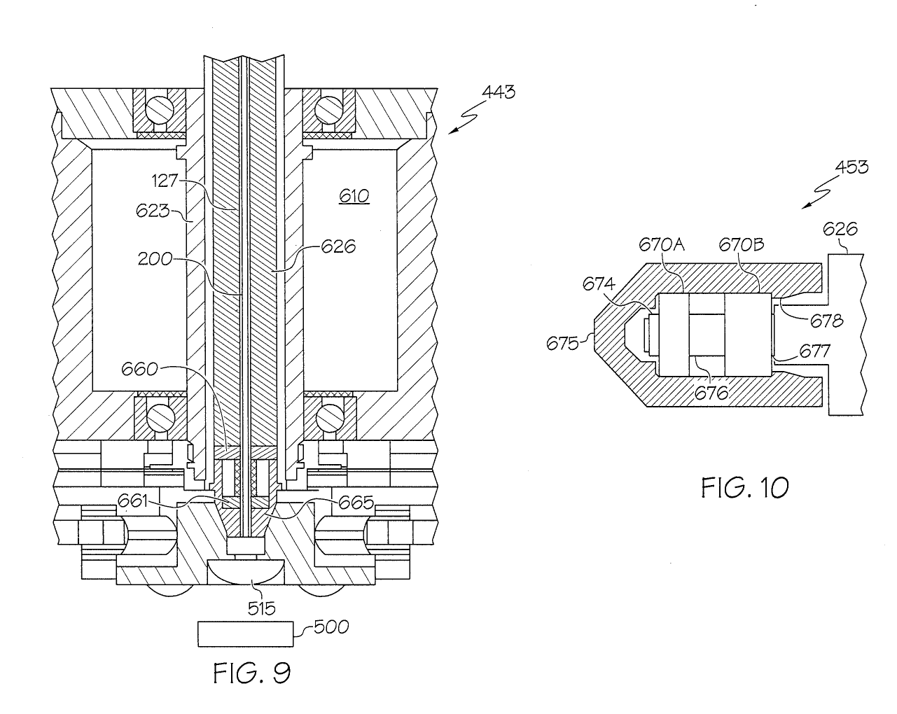

FIG. 9 is a cross-sectional view of a proximal portion 443 of a temperature measurement probe, consistent with the present inventive concepts. The temperature measurement probe may include components that are similar to or the probe 100 described herein, and descriptions thereof are not repeated due to brevity.

Proximal portion 443 of a temperature measurement probe of FIG. 9 is different than those illustrated in FIGS. 6-8 in that proximal portion 443 includes a thrust bearing 660 and a radial bearing 661 between proximal ferrule 665 and probe connector 626. Thrust bearing 660 is constructed and arranged to accommodate a thrust load, for example, during linear movement, and is positioned between a top portion of a stationary proximal ferrule 665 and a rotatable probe connector 626. Radial bearing is constructed and arranged to accommodate a radial load, and is positioned in a cavity or the like in the ferrule 665, and below the thrust bearing 660.

FIG. 10 is a cross-sectional view of a proximal portion 453 of a temperature measurement probe, consistent with the present inventive concepts. The temperature measurement probe may include components that are similar to or the probe 100 described herein, and descriptions thereof are not repeated due to brevity.

Proximal portion 453 may include a proximal ferrule 675 and probe connector 626, similar to those described at least in FIGS. 6-9. Fiber assembly (not shown) is coupled to proximal ferrule 675, and held in a stationary position, similar to embodiments described at least in FIGS. 6-9. Proximal ferrule 675 supports dual radial ball bearings 670A, B. A bushing 676 is coupled to and extends from probe connector 626 to an interior of proximal ferrule 675. The ball bearings 670A, B (generally, 670) or the like are retained in proximal ferrule 675. An annular ridge 678 extends from the proximal ferrule 675, and provides an undercut for the bearings 670A, B to snap into place.

Bearing shim 677 is inserted between distal ball bearing 670B and portion of probe connector 626 inserted in proximal ferrule 675 to separate these elements from each other, and prevent grinding or other undesirable interaction. A retaining shaft snap ring 674 can be included to maintain separation of, and proper positioning of, the bearings 670a, 670b.

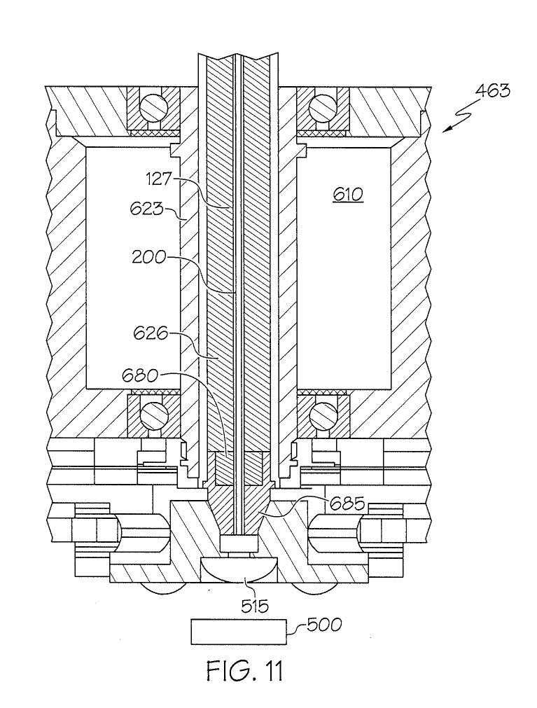

FIG. 11 is a cross-sectional view of a proximal portion 463 of a temperature measurement probe, consistent with the present inventive concepts. The temperature measurement probe may include components that are similar to or the probe 100 described herein, and descriptions thereof are not repeated due to brevity.

Proximal portion 463 of a temperature measurement probe of FIG. 11 is different than those illustrated in FIGS. 6-10 in that proximal portion 463 includes a roller needle bearing 680 between proximal ferrule 685 and probe connector 626.

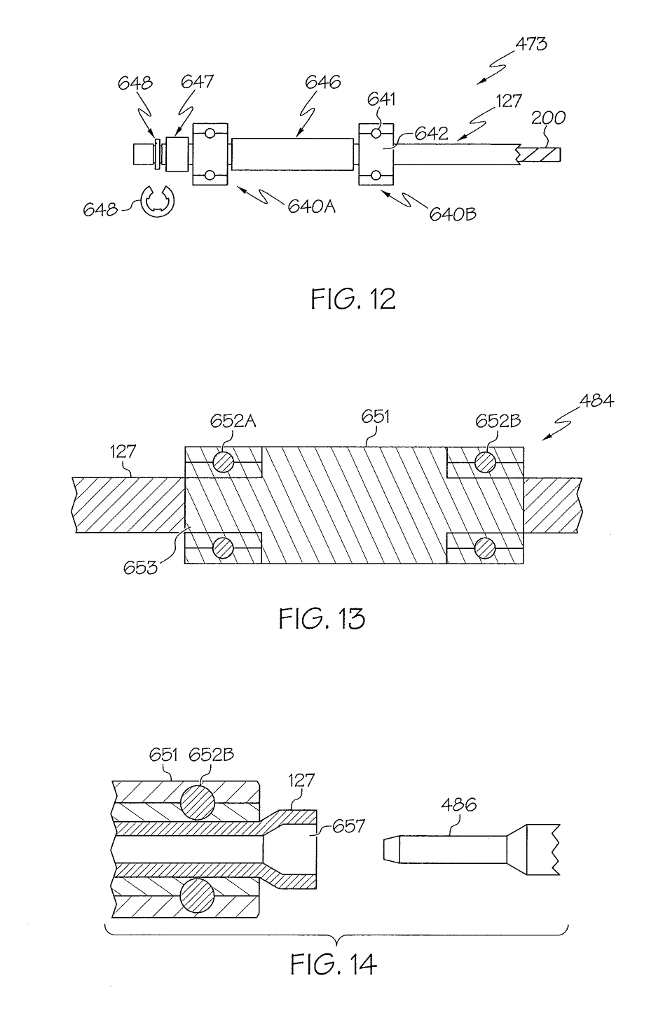

FIG. 12 is a view of a proximal portion 473 of a temperature measurement probe, consistent with other embodiments of the present inventive concepts. The temperature measurement probe may include components that are similar to or the probe 100 described herein, and descriptions thereof are not repeated due to brevity.

Proximal portion 473 of a temperature measurement probe of FIG. 12 is different than those illustrated in FIGS. 6-11 in that proximal portion 473 includes a pair of ball bearings 640A, B, two spacers 646, 647, and a retaining clip 648 at a distal end of the proximal portion 473. Ball bearings 640A, B may be similar to those described in other embodiments, for example, including a ball bearing portion 641 coupled to a stationary conical ferrule (not shown), and an interior portion 642 coupled to a proximal torque coil termination region that permits the torque coil 127 to rotate about fiber assembly 200. The dual bearing configuration provides stability and alignment resulting in a reduction in vibrations originating at the proximal end of the probe and traveling down the length of the device. Retaining clip 648 prevents any sliding or other linear motion of the torque coil 127 inside the stationary ferrule. Spacer 646 maintains separation of ball bearings 640A, B at a predetermined distance from each other. Spacer 647 maintains linear separation of ball bearing 640A from retaining clip 648.

FIG. 13 is a view of a proximal portion 484 of a temperature measurement probe, consistent with the present inventive concepts. The proximal portion 484 includes a single long bearing assembly 651 as an alternative to the ball bearing/spacer configuration illustrated at proximal portion 473 of FIG. 12. Long bearing assembly 651 can include races 652A, B at the ends of the bearing assembly 651, which each couples to a distal ferrule (not shown). Long bearing 651 includes a hollow interior region 653 (FIG. 13 illustrates a cross-section of the long bearing 651) through which torque coil 127 can extend. This configuration permits the torque coil 127 to rotate about fiber assembly 200, while also preventing or minimizing undesirable linear movement of torque coil 127 relative to fiber assembly 200. Bearing assembly 651 provides for easier assembly and high reliability. Also, long bearing 651 provides for improved concentricity between the bearings resulting in smoother operation and less vibration.

As shown in FIG. 14, during manufacture, the bearing 651 can be held in place against the torque coil 127 by a flared tube end 657 which can be formed by a mandrel 486 or the like, in accordance with some embodiments. The torque coil can therefore apply a force against long bearing 651, more specifically, at an end of the long bearing 651 having a race device 652B. The flared end 657 serves as a stop so that long bearing 651 is prevented from sliding linearly along torque coil 127, to provide a function similar to a retaining ring or retaining clip described herein.

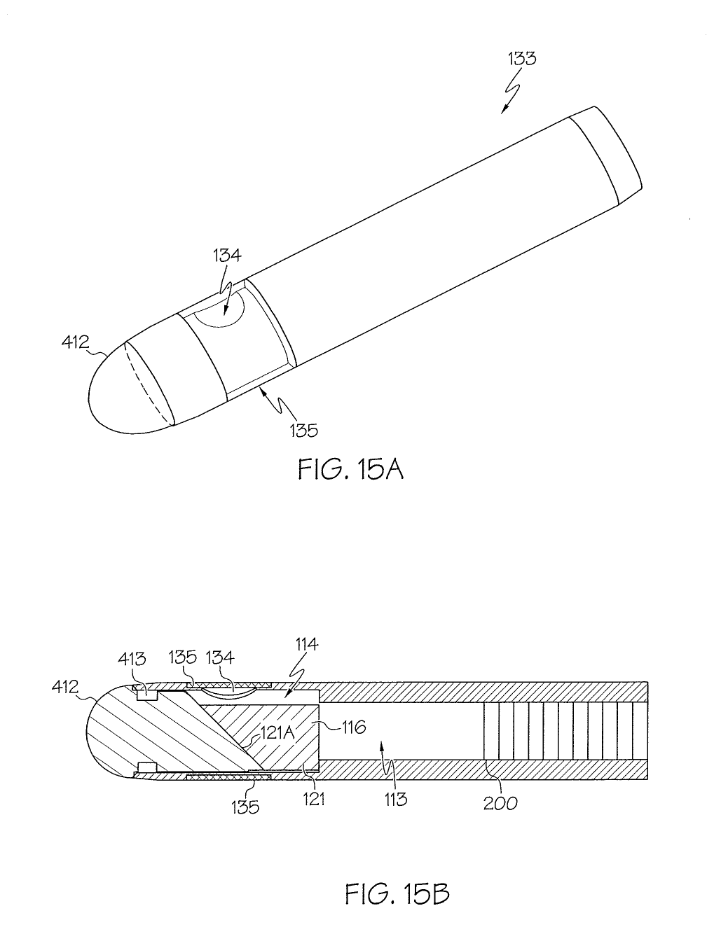

FIG. 15A is a perspective view of an optic sleeve 133, consistent with the present inventive concepts. FIG. 15B is a cross-sectional side view of the optic sleeve 133 of FIG. 15A.

Optic sleeve 133, or holder, is constructed and arranged for housing an optical element 121 positioned at the distal end of a probe assembly. In various embodiments, the optic sleeve 133 can be formed of stainless steel, one or more metals, alloys, composite material, or other material. In various embodiments, the optic sleeve 133 can be machined, molded or otherwise suitably formed.

In some embodiments, the optic sleeve 133 can include a groove on its outer surface to accommodate the positioning of a thin wall extrusion 135, so that an outer surface of the extrusion 135 is aligned or flush with the surface of the sleeve body. In some embodiments, the extrusion 135 is formed of a material that is largely of transmissive of electromagnetic energy in the IR wavelengths, such as low density polyethylene (LPDE) or other transmissive materials. The extrusion 135 can be stretched or heat shrunk over the end of the sleeve 133 to the groove. The sleeve 133 may include a small circular or other shaped aperture 134 that operates as an IR transparent window, for example, in a manner similar to the window 126 described in connection with the embodiment of FIG. 2. In some embodiments, the aperture 134 is aligned with an optical element 121 (see FIG. 15B) or more specifically, a mirror or the like configured to receive and redirect the incident IR light. The aperture 134 may be smaller than the window over which it is positioned, and reduces the concaving effect that a larger window would have. The sleeve 133 may serve partly as a seal, preventing particulates from interfering with the optical element and/or the distal end of the fiber assembly 200. Since the sleeve 133 is driven to rotate and linearly reciprocate as described herein, a rounded tip 412F can be provided at the distal end of the sleeve 133 to prevent the sleeve 133 from cutting into or through, or otherwise damaging, the interior of the distal end region of an external polyethylene sheath or the like positioned in which the tip 412 is positioned. Although the tip 412 is illustrated and described with respect to FIGS. 15A and 15B, it is not limited thereto. The tip 412 may include a coupling mechanism 413 such as one or more tabs that interface with the sleeve body for holding the tip 412 in place. In some embodiments, the aperture 134 may be circular in shape and relatively small as compared to the size of the mirror, which can reduce manufacturing problems associated with LDPE material forming the extrusion 135 from sinking, or having a concaving effect, with respect to the aperture 134.

Optical element 121 may be the same as or similar to an optical element described herein, for example, in FIGS. 3A-3C, which include a reflective surface 121A constructed and arranged to function as a lens to redirect incident IR energy toward a distal end of the fiber assembly 200. In particular, the reflective surface 121A redirects infrared energy incident thereon in a direction transverse a longitudinal direction of the fiber assembly 200 to a distal end of the fiber assembly in the longitudinal direction of the fiber assembly. The end cap 412 of the holder is at a distal end of a longitudinal opening where the reflective surface 121A of the optical element 121 is positioned. In some embodiments, a first portion of the end cap 412 is positioned within the longitudinal opening and a second portion of the end cap 412 extends beyond a distal end of the longitudinal opening. The reflective surface 121A of the optical element 121 may lie at an acute angle relative to a longitudinal axis of the longitudinal opening of the holder and the reflective surface 121A may abut an end surface of a portion of the end cap. In some embodiments, the reflective surface 121A of optical element 121 can be formed of a reflective material, or a reflective coating. In some embodiments, optical element 121 permits light to pass through an IR transmissive material, for example, comprising a germanium, zinc selenide, or related material. In a case where a focusing mirror is employed, such as the embodiments of FIGS. 15A and 15B, this configuration can help to reduce use of relatively expensive IR transmissive materials. Optical element 121 can be separated from a distal end of a fiber 200 by an air gap 113 or medium that provides IR energy to be exchanged between the optical element 121 and the fiber 200. An air gap 114 or related medium may also be positioned between a top surface of optical element 121 and extrusion 135 at aperture 134. In particular, optic element 121 has a flat surface facing the opening 134. IR energy received through the aperture 134 passes through the flat surface of the optical element 121, and is internally reflected within the optical element 121 at a 45 degree angle at reflective surface 121A. Optical element 121 may have a curved output surface 116 that can be employed to further focus the reflected and emitted IR energy on fiber 200.

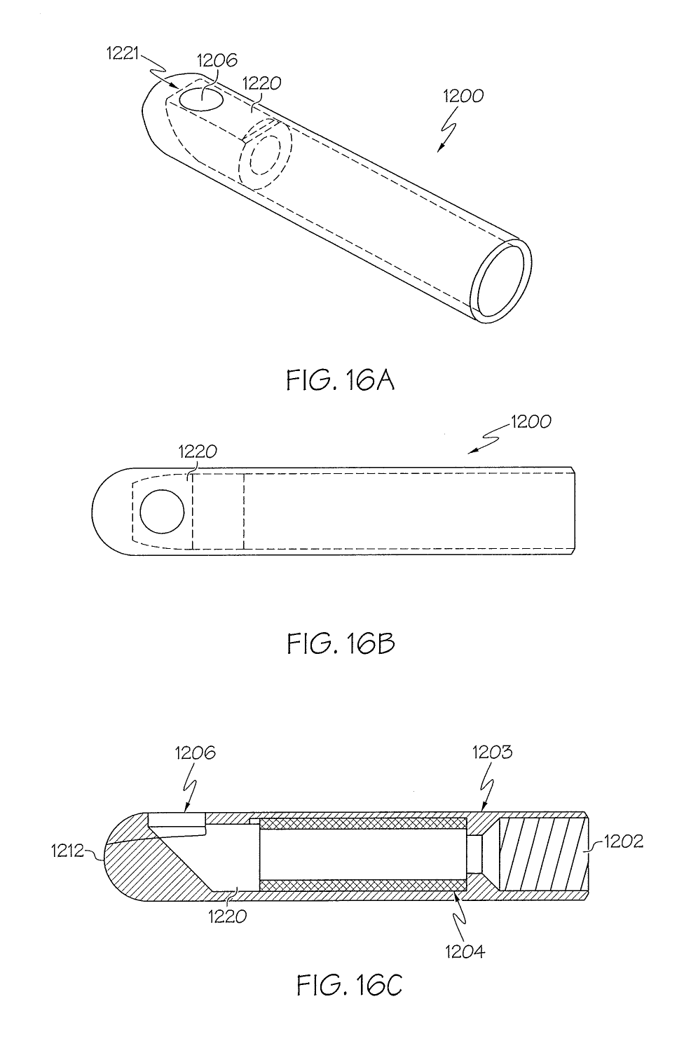

FIGS. 16A-16C are views illustrating a method for enclosing a distal optic 1220 in a molded sleeve 1200, consistent with the present inventive concepts. Sleeve 1200 is constructed and arranged for positioning about a distal end of a probe, for example, similar to optic sleeve 133 described in FIGS. 15A-B. Distal optic 1220 can include a sharp edge 1221. Sleeve 1200 can include a window 1206 that exposes optic 1220 for receiving IR energy from a tissue surface. Window 1206 can be formed of a transmissive material, such as LDPE. Sleeve 1200 can include an undercut 1203 that retains tube 1204 that applies a force against optic sleeve 1200. In this embodiment, tube 1204 can be configured to hold optic 1220 in place. Sleeve 1200 may include a threaded region 1202 for mating with a distal ferrule, for example, described herein, or other probe element.

A rounded tip 1212 is part of the molded distal optic sleeve 1200, and not separate as with the tip 112 illustrated in FIGS. 15A and 15B.

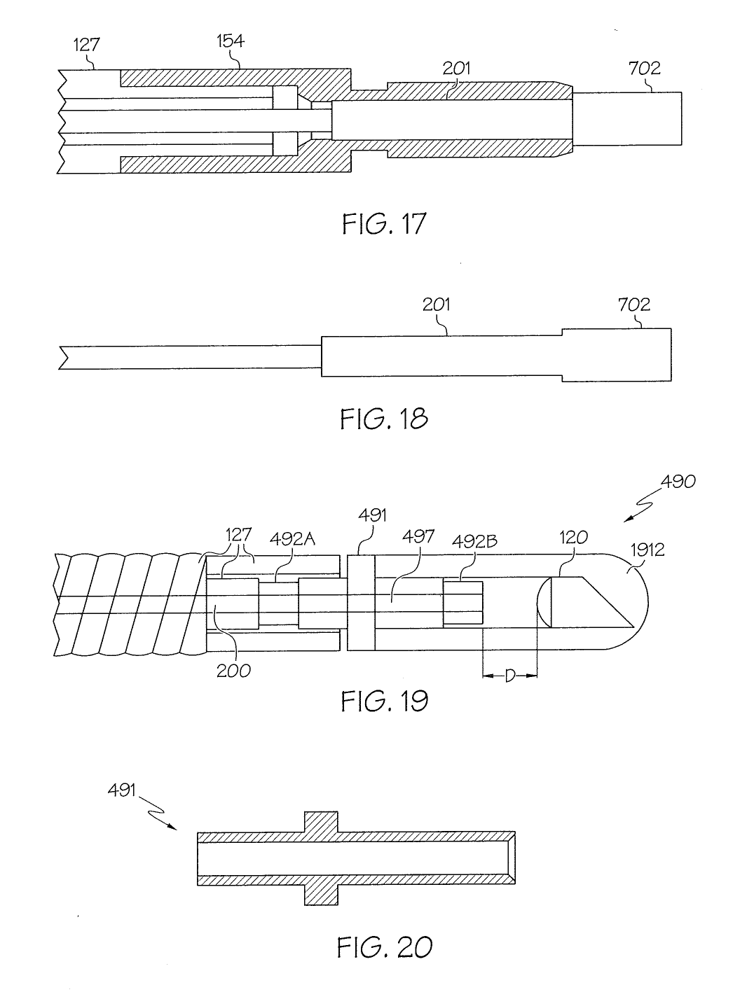

FIGS. 17 and 18 are views of a coupling configuration for coupling a fiber sheath 201 and a distal ferrule 154 for retaining the fiber assembly 200 of a temperature measurement probe, consistent with the present inventive concepts. In the present embodiment, fiber sheath 201 can take the form of a lubricious sleeve, for example the sleeve 128 described herein in connection with the embodiment of FIG. 2. The fiber sheath 201 can be constructed and arranged to surround one or more fibers of fiber assembly 200 described herein. In some embodiments, the fiber sheath 201 operates as a bearing between the body of the rotationally fixed fiber assembly 200 and the rotationally moving surrounding torque coil 127, as described herein.

As described herein in connection with the embodiment of at least FIGS. 2 and 5, maintenance of the distance D between the optical element 121 and the distal end of the fiber assembly 200 to a consistent degree can lead to optimal results. The coupling configuration helps toward maintaining the distance D.

Distal ferrule 154 operates as a mount for the end of the rotationally fixed fiber assembly 200 and fiber sheath 201, and can be similar to a distal ferrule described in other embodiments, for example, distal assembly 312 described in FIG. 5.

As shown in FIG. 17, a fiber sheath bond region 702 is inserted into distal ferrule 154, for example, by pressing or other force applied for moving bonded region 702 into thru-hole in distal ferrule 154. This allows for rotation of torque coil 127 with no translation of fiber assembly.

The fiber sheath 201 is bonded to the fiber to protect in over its length against abrasion but also to protect it from coming in contact with any ferrous materials. The torque coil 127 is formed of steel so the fiber cannot make contact. The fiber sheath bond region 702, often referred to as a button head, can act as a bearing against the distal fiber ferrule 154. When the device is manufactured, the torque coil 127 is compressed so there is a slight load placed on the button head 702 preventing the fiber 201 from moving axially during translation and/or rotation cycles.

FIG. 19 is a cross-sectional view of a distal portion 490 of a temperature measurement probe, consistent with the present inventive concepts. The temperature measurement probe may include components that are similar to or the probe 100 described herein, and descriptions thereof are not repeated due to brevity.

Distal portion 490 includes a fiber protective sheath 497 with first and second heads 492A, B positioned on both sides of distal ferrule 491 for protecting the fiber assembly 200. Also, the distal ferrule 491 allows for rotation of a torque coil 127 and optical element 120 about fiber assembly 200. However, separation between coil 127 and fiber assembly 200 is reduced or eliminated due to the presence of protective sheath heads 492A, B on either side of distal ferrule 491, so that a distance D between distal end of fiber of the fiber assembly 200 and optical element 120 is maintained regardless of any reciprocating motion that may provide forces that attempt to separate the fiber assembly 200 from the optical element 120. Distal ferrule 491 is reduced in length to accommodate both bearings 492A, B. A distal optic holder 1912 is positioned about the optical element 120, the second head 492B and a portion of the distal ferrule 491.

FIG. 20 is a cross-sectional view of the distal optic ferrule 491 of FIG. 19. at a portion of a temperature measurement probe, consistent with the present inventive concepts. Ferrule 491 is constructed and arranged, to prevent fixed attachment between torque coil 127 and fiber assembly 200 during rotation of torque coil 127 about fiber assembly 200. In some embodiments, the length of the fiber may be increased, for example, to accommodate the heads 492A, B shown in FIG. 19. The distal optic sleeve 490 is configured to match the increased length of the fiber assembly 200.

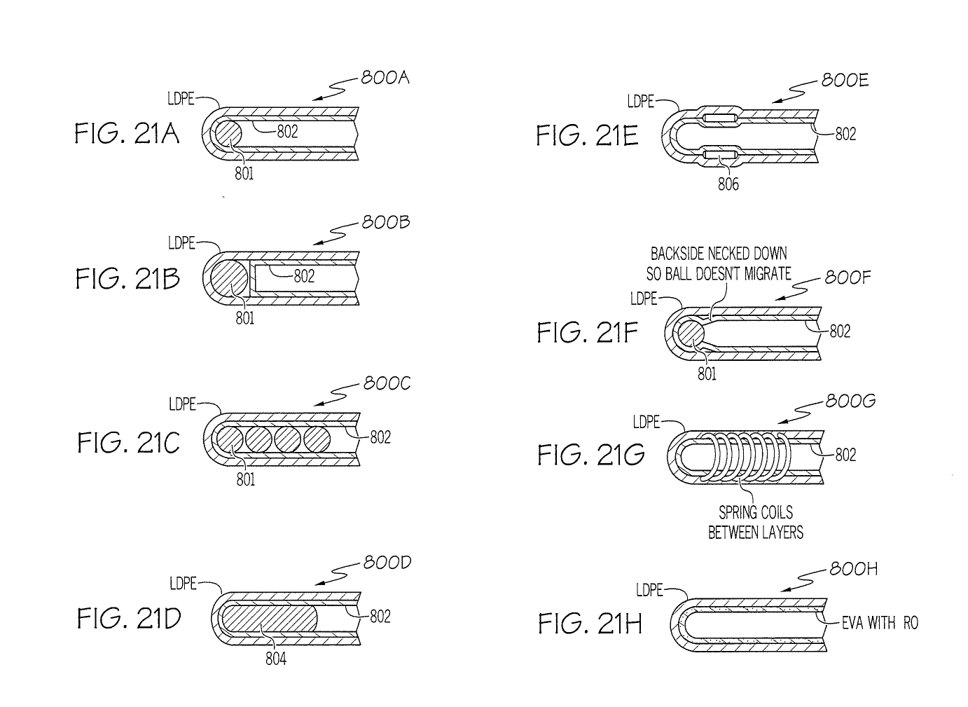

FIGS. 21A-H are cross-sectional views of various radiopaque sheath tips 800A-800H, consistent with the present inventive concepts. The sheath tips 800A-800H (generally, 800) include ball-shaped objects 801 (FIGS. 21A-C, F), pins 804 (FIG. 21D) or the like that are formed of various radiopaque materials, including but not limited to stainless steel (SS) or other radiopaque material or plastic impregnated with radiopaque material. The interior of a sheath tip 800 may be lined with an ethylene vinyl acetate layer, or other soft plastic or the like. In some embodiments, for example, shown in FIG. 21H, the interior may be formed of a radiopaque material, for example, EVA and a radiopaque additive (RO).

The sheath tips 800 may include a marker (FIG. 21E) at the tip of a device. The visibility of the sheath tip 800 by providing a marker is important if the probe folds back on itself during insertion, for example, in situations where failure occurs during an imaging operation where the torque coil may bind.

Other configurations may be provided, such as those shown in FIGS. 21F-21H, but not limited thereto.

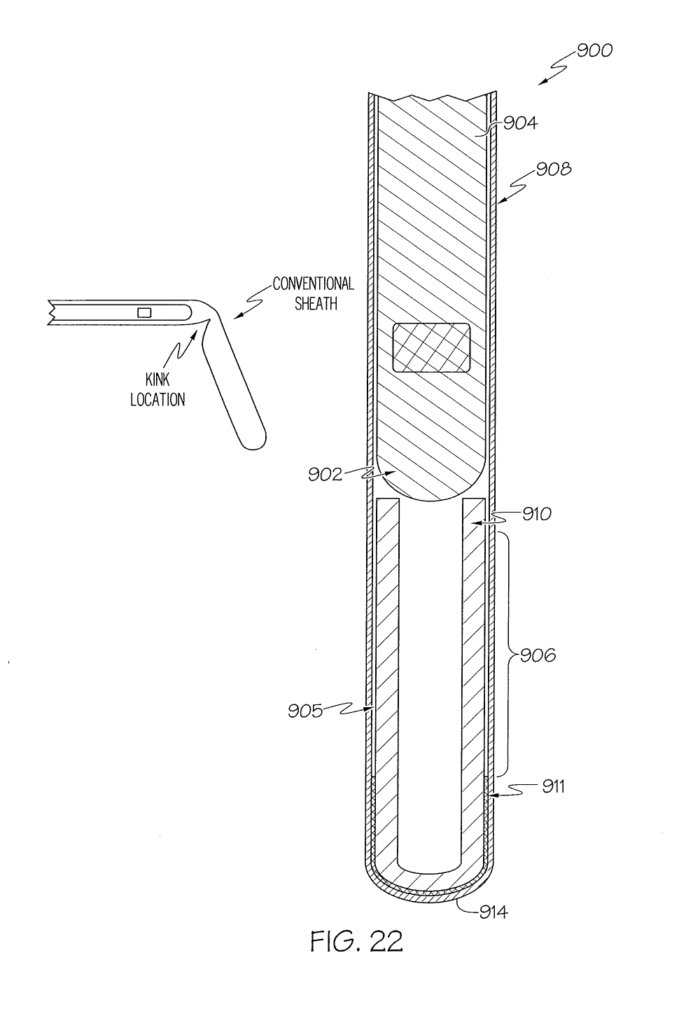

FIG. 22 is a cross-sectional view of a kink-resistant sheath tip 900, consistent with the present inventive concepts. Sheath tip 900 is constructed and arranged to reduce the likelihood of undesirable kinking of a distal end 902 of probe while navigating through a body lumen. A distal end of the probe includes an optical element 902, for example similar to the optical element 121 described herein. In a typical configuration, a longitudinal spacing is present between the distal end of the optical element 902 and the interior of the distal end of the sheath 111. Such spacing leaves a void at which kinking of the sheath 111 can occur.

In the present embodiment, sheath tip 900 includes a first portion 904, a second portion 906, and a third portion 914. The first portion 904 includes a low density extrusion, for example, a polyethylene extrusion (LDPE) 908 or the like, or formed of other materials well-known for forming probe sheaths.

The second portion 906 includes the low density extrusion 908 as the first portion. The second portion 906 also includes a layer of an ethylene vinyl acetate (EVA) extrusion tube 910, or lining, that forms a thick wall inside the LDPE wall 908. The probe tip 902 may be positioned against the EVA extrusion tube 910. The EVA extrusion tube 910 can be U-shaped as shown, or other shape that conforms with the distal end of the sheath tip 900, which may include the second portion 906 and/or third portion 914.

A thin gap 905 may be extend along a portion of the second portion 906 between the LPDE wall 908 and a wall of the EVA tube 910. The third portion 914 may include a thermal fused region 911 that bonds the LDPE wall 908 and the EVA extrusion tube 910. The foregoing configuration therefore provides a reinforcement unit that mitigates kinking at the distal end. The reinforcement unit may further comprise an insert comprising at least one of one or more balls, one or more pins, or a coiled material, or the like.

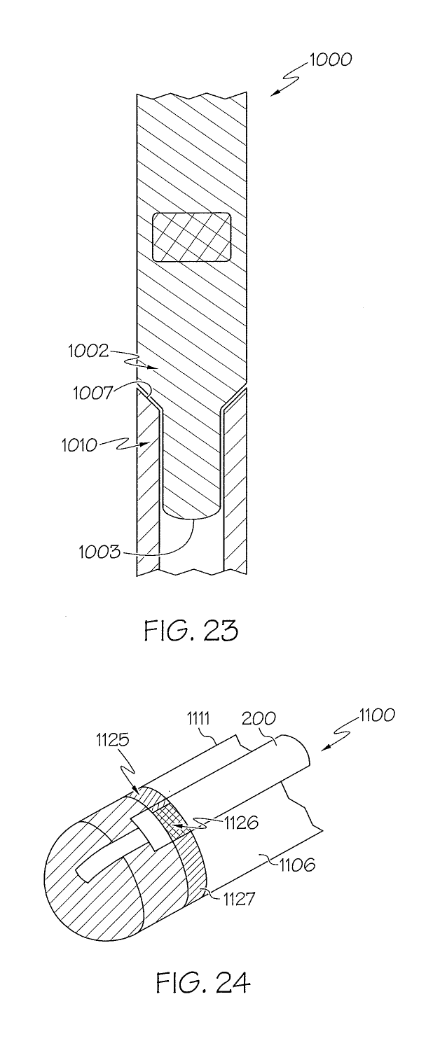

FIG. 23 is a cross-sectional view of another embodiment of a kink-resistant sheath tip 1000, consistent with the present inventive concepts. Elements of sheath tip 1000 can be similar to or the same as sheath tip 900 described in FIG. 22.

Sheath tip 1000 includes an optical element 1002 having an extension tip 1003, or a distal end with a smaller width or diameter than its main body portion. The distal end 1003 of optical element 1002 can be positioned in EVA extrusion tube 1010. The extension tip 1003 in some embodiments may mechanically communicate with a reinforcement unit, for example, illustrated in FIG. 22 or 23. A proximal end of EVA extrusion tube 1010 may include a bevel or chamfer 1007 for receiving the distal end 1003 of optical element 1002, which may offer additional kink resistance for probe, in particular during translation and/or rotation of probe distal end 1003 relative to sheath tip 1000.

FIG. 24 is a perspective view of a probe 1100 configured to include a multi-toned marker band 1125 about its sheath 1111, in accordance with some embodiments. Although one marker band 1125 is shown, sheath 1111 is part of a probe shaft 110 that may optionally include two or more marker bands, which can be placed over and/or adjacent to the proximal and distal ends of window 1106 which permits IR data to be received during a temperature measurement operation from tissue visible through the window 1106. Bands 1125 can be visualizable or identified such as to aid in positioning probe. Bands 1125 can comprise a material selected from the group consisting of: a radiopaque material; aluminum, titanium, gold, copper, steel, iridium, platinum cobalt, chromium; and combinations of these and/or a material with a known emissivity, such that fiber assembly 200 records the infrared temperature information of bands 1125 when infrared light emitted from a band 1125 is received by fiber assembly 200. Bands 1125 can be constructed and arranged such that when a collector, such as distal end of fiber assembly 200 is positioned within band 1125 (e.g. collects infrared light transmitted from band 1125), a signal is received by sensor assembly 500 comprising a pre-determined or otherwise separately measurable signal, such as a pre-determined pattern of infrared reflectance or emissivity, or a measurable temperature.

Band 1125 can include one or more temperature sensors, such as one or more thermocouples, thermistors, or other temperature sensors, which can be configured to measure temperature information of band 1125 proximate one or more tissue locations. Marker band 1125 is positioned in a similar manner as in other embodiments, for example, circumferentially about the sheath 1111. The inner surface of marker band 1125 may include a first region 1126 and a second region 1127 formed differently from each other, and more importantly, has different and known emissivities. In some embodiments, the first region 1126 is formed of a different material than the second region 1127. In other embodiments, the first region 1126 has a different color than the second region 1127.

The second region 1127 may be smaller than the first region 1126. Although a two-tone marker band (1126, 1127) is shown, other configurations can equally apply, such as one or more marker bands having more than two regions, colors, materials, or other features for distinguishing the regions from each other. As the interior of the band 1125 is imaged during a temperature measurement operation, the different emissivities will appear as two temperatures with respect to an IR detector. The resultant change in temperature as perceived by the IR detector will be a known constant. The slope of the system can therefore be calculated directly, for example, used to perform temperature measurement as described herein.

For example, a collection region at the distal end of fiber assembly 200 is at region 1126, whereby detector can indicate a different temperature region than the temperature reading at the rest of the circumference at region 1127 of the marker band 1125. Therefore, a sensor can, and a display can display that distal end of the fiber assembly 200 has collected IR data through the IR transmissive region 1126, which may provide a reference point.

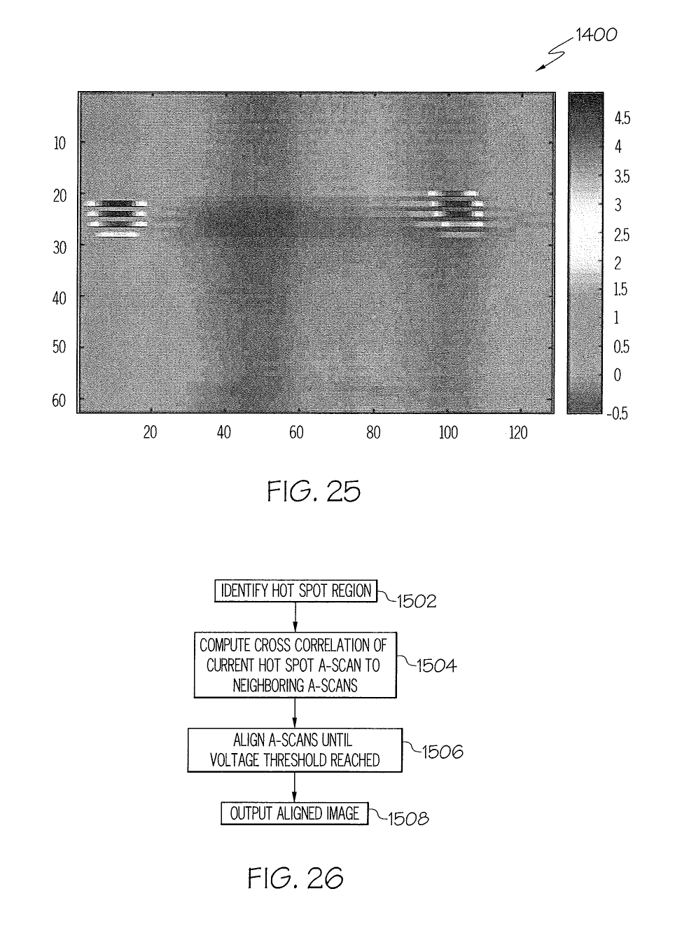

FIG. 25 is an image 1400 of a scan result illustrating a misaligned hot spot, which is addressed by a temperature measurement probe, consistent with some present inventive concepts. FIG. 26 is a method for realigning A-scans of a hot spot region, consistent with some present inventive concepts.

A described above, a temperature mapping system In some embodiments, includes a rotary motor that is constructed and arranged to rotate torque coil 127 which in turn rotates optical assembly 120 relative to a fiber assembly during a temperature measurement operation. This may include the probe being positioned in a body lumen performing a rotational scan, referred to herein as an A-scan of a cross-section of a tissue surface region about the region. An A-scan on a single 360° line may include many individual temperature readings. In some embodiments, 128 samples are taken in a scan spinning at 3600 RPM, but not limited thereto. The probe assembly can also perform a translational B-scan along a length of an IR transmissive region of a probe, for example, at a proximal end of the probe sheath relative to a marker band or opaque region, or between two marker bands. A B-scan is the compilation of all the A-scans required to make a full translation over a predetermined length, for example, 60 mm. For example, the probe can translate 60 mm/sec so there are 60 A-scans in every B-scan. During the A-scan or the B-scan, multiple IR energy readings may be taken from a surface of a body lumen in which the probe is positioned. A processor such as signal processing unit 400 described with respect to FIG. 1 can process information signals converted by a sensor, for example, sensor assembly 500. User interface 300 may output the scan results in graphical form, i.e., a temperature map. The temperature map correlates to the geometry of the multiple collection location results of the probe scan, and is a representation of the temperature profile of the “unfolded” luminal wall or other body tissue.

However, a rotary motor may be prone to variability in rotational speed, which can cause a misalignment in the positioning of the resulting A-scans, for example, shown in FIG. 25 as two distinct hot spot images. Thus, a hot spot may appear scattered across A-scans, which may confuse a viewer.

In sum, the system in accordance with some embodiments rotates A-scans to align a hot spot.

At step 1502, a general hot spot region is identified in the image. An image processing technique may be performed to identify a hot spot region. For example, an image segmentation process may be performed that identifies a hot spot region relative to a background region.

For example, a probe scan during an A-scan or a B-scan may reveal a hot spot indicating that a region of the body lumen of interest has a temperature that is beyond (above or below) a desired temperature range, or is higher (or lower) than a temperature of other regions of the body lumen, which can be displayed.

At step 1504, a cross-correlation is computed between the current hot spot A-scan to neighboring A-scans, in order to realign the A-scans, for example, to identify an alignment position with respect to an A-scan.

At step 1506, the A-scans are aligned until a voltage threshold is reached. At step 1508, the aligned image is output for display.

User interface 300 can display a temperature key along with the hot spot for associating the displayed colors of the temperature map to the correct temperature. A graph can also be displayed, which depicts the probe A-scan results in a graphical form in addition to or instead of temperature map. In an analogous arrangement, temperature gradients, rates of change in time or space, can be depicted in the display fields as a function of time and in the color-mapping key. As such, the rate of change of temperature and the peak rate of change in temperature, or other parameters can be continuously determined and conveyed to the user.

In connection with the embodiment of the present inventive concepts, while the term “hot-spot” is used to identify a region of significance on the image, for purposes of the present inventive concepts, the term applies equally well to other regions of interest, such as a hot or cold temperature region, or a region having a relatively rapid change of temperature in time or space.

In some embodiments, two image processing techniques are combined to identify a hot spot region and realign the A-scans. First, an image segmentation process referred to as region growing is adapted to identify the hot spot region in the image. Second, template matching, or cross correlation, is used for realigning A-scans. A special purpose processor, for example, a hardware processing device, performs some or all of the process.

The hot spot region and background region are identified. An estimate of a background rotationally induced signal (RIS) is determined, for example, a median of background A-scans. The region growing process is initialized to start at the peak A-scan of the hot spot region. A-scans are added to the hot spot region based on peak voltage (after subtracting off updated background estimate). A cross correlation of a current hot spot A-scan to neighboring A-scans is computed to identify an alignment position. The process is repeated to expand the hot spot region and align A-scans until a voltage threshold is reached. A final estimate of an RIS background signal is computed for monitoring. An aligned image is output for display.

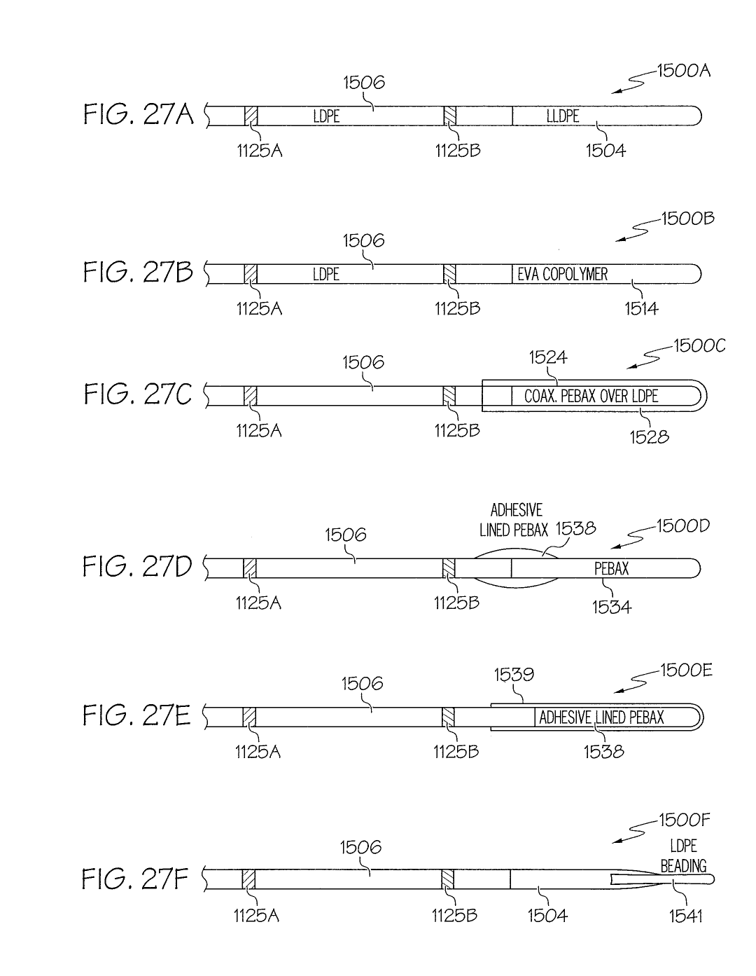

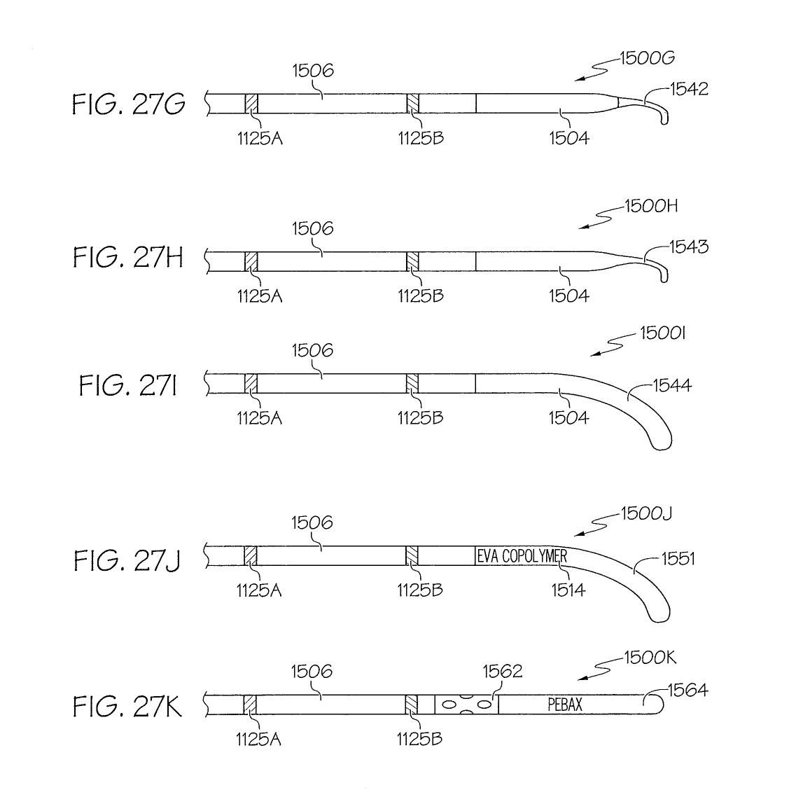

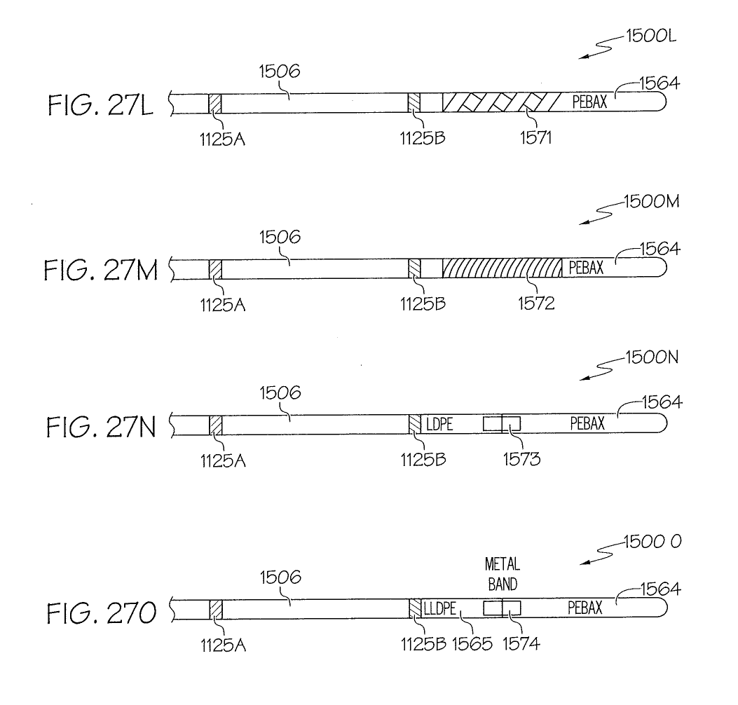

FIGS. 27A-27O are views of embodiments of different configurations of a distal end of a probe, consistent with some present inventive concepts. Some or all of the probe tips have a distal end that may be formed using a mandrel, heating, or other formation techniques.

As shown in the embodiment of FIG. 27A, distal end 1500A of probe includes a window segment 1506 formed of LDPE or the like positioned between a proximal marker band 1125A and a distal marker band 1125B. The proximal and distal marker bands 1125A, 1125B (generally, 1125) are preferably coupled to both sides of the LPDE window segment 1506. At the outermost end 1504 of the probe sheath is formed of linear low-density polyethylene (LLDPE) or the like coupled to the LDPE window segment 1506, which has a wall having a smaller thickness than the LDPE window segment 1506.

As shown in the embodiment of FIG. 27B, distal end 1500B of probe includes an LDPE window segment between two marker bands 1125A, 1125B, similar to FIG. 27A. However, the outermost end 1514 of the probe sheath is formed of flexible ethylene copolymer material, e.g., EVA, or the like.

As shown in the embodiment of FIG. 27C, distal end 1500C of probe includes an LDPE window segment 1506 between two marker bands 1125A, 1125B. The outermost end 1524 of the probe sheath is also formed of LDPE, so that the sheath including both the window segment 1506 and outermost distal segment 1524 are formed from a same material, i.e., LDPE. However, a coextrusion 1528 of a Pebax material can be formed over the LDPE sheath at the distal segment. The LDPE has a thickness so as to permit the Pebax to determine the performance of the segment.

As shown in the embodiment of FIG. 27D, distal end 1500D of probe includes an LDPE window segment 1506 between two marker bands 1125A, 1125B. However, unlike FIG. 27C, the outermost distal segment 1534 is formed of a flexible material, namely, Pebax or the like. The Pebax distal segment is coupled to the LDPE segment by an adhesive lined segment 1538, which may include Pebax or the like. The adhesive lined segment 1538, or bonding region, may have a diameter that is greater than the coupled LDPE window 1506 and Pebax 1534 segments.

As shown in the embodiment of FIG. 27E, both the outermost distal segment and the adhesive lined segment 1538 of a probe 1500E are formed with a low durometer adhesive lined Pebax 1539 with an adhesive inner surface that bonds to the LDPE segment 1506, in particular, a portion of the LDPE segment external to the window segment 1506, and distal from the distal marker band 1125B.

As shown in the embodiment of FIG. 27F, a beading tip 1541 may be coupled to an LLDPE segment 1504 at the outermost distal end 1500F of the probe sheath, for example, shown in FIG. 27A. The beading tip 1541 can be fuse heated to the LLDPE segment 1504, providing flexibility while also adding additional length to the distal end 1500F.

As shown in the embodiment of FIG. 27G, a tip 1542 formed of flexible EVA copolymer or the like may be coupled to an LLDPE segment 1504 at the outermost distal end 1500G of the probe sheath, for example, shown in FIG. 27A. The tip 1542 may be tapered. The tapered tip 1542 may include a curve or other shape allowing the tip 1542 to be used to navigate a nasal cavity or other body orifice. This region 1542 is formed of a softer material than the LLDPE segment 1504.

As shown in the embodiment of FIG. 27H, LLDPE segment 1504 at the outermost distal end 1500H of the probe sheath includes a curved end 1543 or other shape allowing the tapered tip to be used to navigate a nasal cavity or other body orifice. Accordingly, the curved end 1543 is part of the LLDPE segment 1504 and formed of the same materials as LLDPE segment 1504.

As shown in the embodiment of FIG. 27I, LLDPE segment 1504 at the outermost distal end 1500I of the probe sheath may be shaped by heat treatment of the like. The heat shaped tip may be used to assist with navigation through a nasal cavity or other body orifice. The curved end 1544 of the LLDPE segment 1504 may have a constant dimension, for example, same or similar diameter or width distinguished from the tapered curve end 1543 of the distal end 1500H illustrated in FIG. 27H.

The embodiment of FIG. 27J may be similar to that of FIG. 27I, except that the distal end segment of the distal end 1500J of the probe is formed of a flexible copolymer 1551, similar to FIG. 27B.

As shown in the embodiment of FIG. 27K, outermost segment 1564 of distal end 1500K of the probe sheath is formed of Pebax or the like. The Pebax distal segment 1564 is coupled to the window segment 1506 by a mechanical joint 1562. For example, a mechanical joint 1562 may include a perforation at the bonding region for coupling the Pebax distal segment 1564 to the window segment 1506.

As shown in the embodiment of FIG. 27L, outermost segment 1564 of distal end 1500L of the probe sheath is formed of Pebax or the like. The Pebax distal segment 1564 is coupled to the window segment 1506 by a mechanical joint 1571. For example, the Pebax tip may form a mechanical joint 1571 after being heat fused to a spiral cut end of the window segment 1506.

As shown in the embodiment of FIG. 27M, distal end 1500M includes an outermost segment 1564 coupled to the window segment 1506 by a mechanical joint 1572 formed by heat-fusing the Pebax tip to a spiral cut end of the window portion 1506. A coil or the like can be formed at the bonding region 1572 between the Pebax tip 1564 and the window segment 1506.

As shown in the embodiment of FIG. 27N, distal end 1500N includes an outermost distal segment 1564, e.g., formed of Pebax or the like, to be coupled to a window segment 1506, e.g., formed of LDPE or the like, by a mechanical joint 1573 including a metal band that forms a thermal bond between the metal band, the flexible Pebax tip 1564, and the LDPE portion of the window segment 1506.

The embodiment of FIG. 27O may be similar to the embodiment of FIG. 27A, except that distal end includes a Pebax distal segment 1564 coupled to an LLDPE stiffness transition segment 1565 by a mechanical joint 1574 including a metal band that forms a thermal bond between the metal band, the flexible Pebax tip 1564, and LLDPE portion 1565.

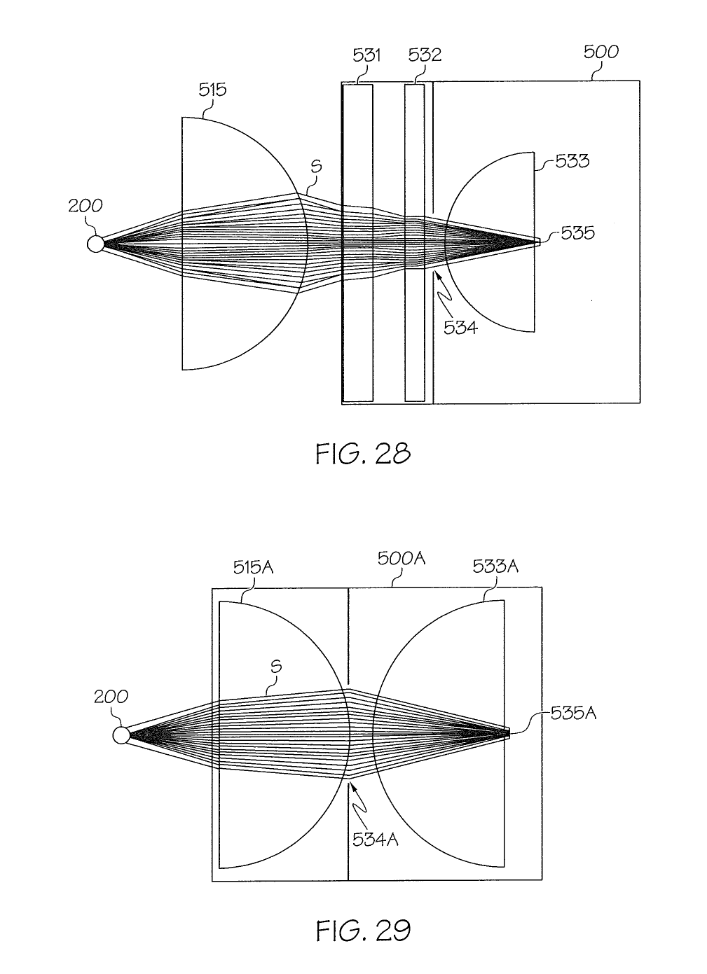

FIG. 28 is a view of a proximal region of a temperature mapping system of FIGS. 1 and 6-11, consistent with some present inventive concepts. The sensor assembly 500 may include but not be limited to a window 531, filter 532, immersion lens 533, and cold stop aperture 534, which collectively receive an output signal from the proximal end of the fiber assembly 200 and focus the energy onto the sensor plane 535. The window 531, filter 532, immersion lens 533, cold stop aperture 534 and sensor plane 535 are well-known to those of ordinary skill in the art, and are not described in detail for reasons related to brevity. Focusing lens 515 may focus light output from fibers of the fiber assembly 200 onto these elements of the sensor assembly 500.

As shown, the focusing lens 515 is external to the sensor assembly 500 and forms the optic path to the sensor assembly 500. The presence of multiple surfaces of the window 531 and filter 534 as well as the materials forming these elements 531, 534 may contribute to a loss of energy as the output signal including light reflects and passes through these elements of the sensor assembly 500 to a sensor plane 535A on the opposite side of the immersion lens 533 which may process the received output signal.

FIG. 29 illustrates an integrated assembly 500A that includes a housing 530, in which is positioned a focusing lens 515A and an immersion lens 533A separated by a predetermined distance. A cold stop aperture 534A may be between the focusing lens 515A and an immersion lens 533A. The interior of the housing 530 may include a vacuum environment. The elements in the housing 530 may be exposed to cold temperatures for improving the path for the signal (S) output from the fiber 200 to the sensor plane 535A in the sensor assembly. The integration of the focusing lens into the window and absence of the filter in the integrated housing 530, and thereby the removal of four surfaces corresponding to the window and filter, respectively, permits a reduction in loss of energy as the light of the output signal (S) reflects and passes through the integrated assembly 500A to the sensor face 535A. The preservation of energy in this manner by eliminating these surfaces may be used to overfill the sensor plane 535A, thereby making the system more tolerant to probe-to-probe alignment with the sensor assembly 500. The system is therefore more tolerant to normal manufacturing tolerances between different probes used in the same patient interface unit 600 (see FIG. 1). The configuration of the integrated assembly 500A also simplifies manufacturing of the patient interface unit 600 because only the fiber assembly 200 needs to be aligned to the detector in the sensor assembly 500A.

While embodiments of the devices and methods have been described in reference to the environment in which they were developed, they are merely illustrative of the principles of the inventive concepts. Modification or combinations of the above-described assemblies, other embodiments, configurations, and methods for carrying out the inventive concepts, and variations of aspects of the inventive concepts that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herein not be construed as being order-specific unless such order specificity is expressly stated in the claim.

As will be appreciated by one skilled in the art, aspects of the present inventive concepts may be embodied as a system, method, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Computer program code for carrying out operations for aspects of the present invention may be written in any combination of one or more programming languages. The program code may execute entirely on the user's computer, partly on the user's computer, as a stand-alone software package, partly on the user's computer and partly on a remote computer or entirely on the remote computer or server.

1. A system that produces temperature estimations of a tissue surface, comprising:
a base including a motion unit;
a fiber assembly including at least one fiber constructed and arranged to receive infrared energy from the tissue surface, the fiber assembly transmissive of infrared energy; the fiber assembly including a proximal end, a distal end and a body;
an optical element that redirects received infrared energy to the distal end of the fiber optic; and
a linkage coupled between the base and the optical element, the fiber extending through the linkage, the linkage coupled to the motion unit at a proximal end and the optical element at a distal end, the motion unit constructed and arranged to rotate the linkage about the fiber assembly to thereby rotate the optical element at the distal end.

2-217. (canceled)
